(19) United States
(12) Patent Application Publication
(10) Pub. No.: US 2012/0022232 A1
(43) Pub. Date: Jan. 26, 2012

(54) CELLS FOR INCREASED PROTEIN EXPRESSION COMPRISING ONE OR MORE RELEASE FACTORS AND METHODS OF USE

(75) Inventors: Rachel Green, Baltimore, MD (US); Hani S. Zaher, Baltimore, MD (US)

(73) Assignee: THE JOHNS HOPKINS UNIVERSITY, Baltimore, MD (US)

(21) Appl. No.: 13/132,528

(22) PCT Filed: Dec. 2, 2009
(86) PCT No.: PCT/US09/66346

§ 371 Date: Aug. 5, 2011

Related U.S. Application Data
(60) Provisional application No. 61/119,204, filed on Dec. 2, 2008.

(51) Int. Cl. C07K 14/00; C12P 21/00; C12N 1/19; C12N 7/01; C12N 1/00; C12N 1/21

(52) U.S. Cl. 530/350; 435/243; 435/252.33; 435/254.21; 435/254.23; 435/235.1; 435/69.1; 435/325; 435/252.1; 435/254.11

(57) ABSTRACT

The invention provides recombinant cells or recombinant restrictive cells for overexpressing one or more proteins comprising one or more release factors. The invention also provides methods for producing a recombinant protein comprising culturing the cells of the present invention under conditions such that one or more proteins are overexpressed, and then isolating the expressed recombinant protein, In certain embodiments of the present invention, the gene encoding the protein is codon optimized.

### CROSS-REFERENCE TO RELATED APPLICATION

This application claims the benefit of U.S. Provisional Application No. 61/119,204, filed on Dec. 2, 2008, the entire contents of which are incorporated herein by reference.

### BACKGROUND OF THE INVENTION

Great strides have been made in the expression of proteins for structure and characterization studies, as well as for therapeutic and diagnostic uses. Generation of recombinant proteins in heterologous systems such as E. coli and insect cells have become important methods for supplying proteins to support structure based drug design. While these systems are generally reliable, many obstacles still remain in utilizing these expression systems to their full potential. Higher throughput expression and purification, as well as more flexible expression systems and techniques are in even greater demand now to support the needs of the research pipeline.

One of the challenges facing in protein expression is the development of a robust and high fidelity system that delivers a quality protein product. The present invention provides a cells and methods for increased quantity and/or quality of protein expression.

### SUMMARY OF THE INVENTION

As described in more detail below, the present invention has identified a new form of quality control on the ribosome that results in the abortive termination of protein synthesis following the misincoporation of amino acids during protein synthesis. This finding suggests that by evading this control mechanism in the cell, protein production can be increased in organisms where this mechanism exists through a variety of approaches.

Present methods of protein expression utilize various approaches to optimize codon choice relative to measured tRNA abundance; however these approaches only work some of the time. Because truncated protein products have not previously been observed, there has been no understanding of the existence of this system. The present invention describes, generally, manipulation of the fidelity of the system through mutations in the ribosome, through drugs, through tRNA abundance, and through the removal or addition of other translation components, such as the release factors RF1 RF2 or RF3, and that will increase the yield of full length proteins.

Accordingly, in one aspect, the invention generally provides a recombinant cell for overexpressing one or more proteins comprising one or more release factors.

In one embodiment, the cell is prokaryotic.

In another embodiment, the prokaryotic cell is an E. coli cell.

In a further embodiment, the cell is a eukaryotic cell.

In another related embodiment, the eukaryotic cell is selected from the group consisting of a bacterial, yeast or mammalian cell. In specific embodiments the cell is a Saccharomyces cerevisiae, Pichia pastoris or a Baculovirus cell.

In another aspect, the invention features a recombinant restrictive cell for overexpressing one or more proteins comprising one or more release factors.

In one embodiment, the cell is selected from the group consisting of: US157, UK285 and UK317.

In one embodiment of the above aspects, the release factor is selected from the group consisting of: release factor 1 (RF1), release factor 2 (RF2), and release factor 3 (RF3).

In one embodiment, the release factor is RF 1.

In one embodiment, the release factor is RF2.

In one embodiment, the release factor is RF3.

In a related embodiment, RF1 corresponds to SEQ ID NO: 1.

In another related embodiment, RF2 corresponds to SEQ ID NO: 2.

In another further related embodiment, RF3 corresponds to SEQ ID NO: 3.

In one embodiment, the release factor is RF2 and RF3.

In another aspect, the invention features a recombinant cell for overexpressing one or more proteins comprising RF3.

In yet another aspect, the invention features a recombinant restrictive cell for overexpressing one or more proteins comprising RF3.

In still another aspect, the invention features a recombinant cell for overexpressing one or more proteins comprising RF1.

In another aspect, the invention features a recombinant restrictive cell for overexpressing one or more proteins comprising RF1.

In yet another aspect, the invention features a recombinant cell for overexpressing one or more proteins comprising RF2.

In still another aspect, the invention features a recombinant restrictive cell for overexpressing one or more proteins comprising RF2.

In another aspect, the invention features a recombinant cell for overexpressing one or more proteins comprising RF2 and RF3.

In yet another aspect, the invention features a recombinant restrictive cell for overexpressing one or more proteins comprising RF2 and RF3.

In one embodiment, the recombinant cell of the above aspects further comprises a vector comprising a codon optimized gene encoding the protein.

In another embodiment, in the recombinant cell of the above aspects post-peptidyl transfer quality control is increased.

In a further embodiment, in the recombinant cell of the above aspects the fidelity of tRNA selection is increased.

In another further embodiment, in the recombinant cell of the above aspects the rate of incorporation of amino acids is suppressed.

In one embodiment, the invention features a method for producing a recombinant protein comprising culturing the cell of the above aspects under conditions such that one or more proteins are overexpressed, isolating the expressed recombinant protein.

In another embodiment, the invention features a method for producing a recombinant protein comprising culturing the cell of claim 5 under conditions such that one or more proteins are overexpressed and isolating the expressed recombinant protein.

In one embodiment of the above aspects, the method further comprises the step of transfecting the cell with a gene encoding the protein.

In one embodiment of the above aspects, the method further comprises the step of transforming the cell with a gene encoding the protein.

In another embodiment, the gene encoding the protein is codon optimized.

In a further embodiment, the gene encoding the protein is codon optimized.

In another aspect, the invention features a method for producing a recombinant protein comprising transfecting or transforming a cell comprising one or more release factors with a gene encoding the recombinant protein, culturing the cell, isolating the expressed recombinant protein.

In one embodiment, the cell is a restrictive cell. In a related embodiment, the restrictive cell is selected from the group consisting of: US157, UK285 and UK317.

In one embodiment, the cell is prokaryotic. In a related embodiment, the prokaryotic cell is an E. coli cell.

In one embodiment, the cell is a eukaryotic cell.

In another embodiment, the release factor is selected from the group consisting of release factor 1 (RF1), release factor 2 (RF2), and release factor 3 (RF3).

In one embodiment, the release factor is RF1.

In one embodiment, the release factor is RF2

In one embodiment, the release factor is RF3.

In one embodiment, the release factor is RF2 and RF3.

In another embodiment, the invention features a recombinant protein produced by the method of any one of the above aspects.

Other features and advantages of the invention will be apparent from the detailed description, and from the claims.

### DEFINITIONS

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3,4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

As used herein, the following terms have the meanings ascribed to them below, unless specified otherwise.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

By “culturing” is meant to refer to maintaining or growing a cell or cells in vitro. Methods of culturing cells are known in the art (see, e.g., Tissue Engineering Methods and Protocols, Morgan and Yarmush (eds.), Humana Press, Inc., Totowa, N.J., 1999). As one ordinarily skilled recognizes, the conditions under which cells are cultured varies depending on the cell type. The conditions include temperature of the environment, the culturing vessel containing the cells, the composition of the various gases, e.g., CO.sub.2, which comprises the cell culture atmosphere or environment, the medium in which the cells are maintained, the components and pH of the medium, the density at which cells are maintained, the schedule by which the medium needs to be replaced with new medium, etc. These parameters are often known in the art or can be empirically determined.

By “overexpress” is meant an increase. An increase can refer to a 5% increase in protein expression, and can encompass an increase of 10%, 20%, 50%, 75%, 100%, 150% or more.

By “release factor” is meant to refer to a protein that allows for the termination of translation by recognizing the termination codon or stop codon in a mRNA sequence. In certain embodiments, a release factor is meant to refer to RF1, RF2 or RF3. Prokaryotic translation termination is mediated by three release factors: RF1 RF2 and RF3. RF1 recognizes the termination codons UAA and UAG. RF2 recognizes UAA and UGA. RF3 is a GTP-binding protein that is normally involved in recycling of the translational machinery. Eukaryotic translation termination similarly involves two release factors: eRF1 and eRF3. eRF1 recognizes all three termination codons. eRF3 is a ribosome-dependent GTPase that helps eRF1 release the completed polypeptide.

By “recombinant cells or cell” is meant to refer to one or more individual cells as well as to a recombinant cell in which the cells are expressing a heterologous protein.

By “transforming” or “transformation” is meant to refer to the transfer of a nucleic acid fragment into the genome of a host cell, resulting in genetically stable inheritance. A “host cell” is a cell that has been transformed, or is capable of transformation, by an exogenous nucleic acid molecule. “Transformed”, “recombinant”, “transduced”, “transgenic”, refer to a host cell or organism into which a heterologous nucleic acid molecule has been introduced. The nucleic acid molecule can be stably integrated into the genome and detected by methods generally known in the art and are disclosed in Sambrook and Russell, infra. See also Innis et al. (1995); and Gelfand (1995); and Innis and Gelfand (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

### BRIEF DESCRIPTION OF THE DRAWINGS

FIG. 1 (a-f) shows unusual behavior of RF2 after a miscoding event. Panel a is a schematic of the core steps of elongation and termination on the ribosome. During the elongation cycle, a ternary complex comprising aminoacylated tRNA, EF-Tu and GTP enters the A site, and reacts with the peptidyl-tRNA elongating the nascent peptide by one amino acid. When a stop codon enters the A site, it is recognized by a class I release factor resulting in the hydrolysis of peptidyl-tRNA. Panel b is a schematic representation of the matched MKX and mismatched MNX (mismatched base pair shown in red) dipeptidyl-tRNA ribosome complexes (dipeptidyl-RNCs), which have a stop codon in the A site. Panel c shows rate constants for release (k_{hyd}) measured on the P-site-matched (MKX) and mismatched (MNX) complexes with saturating RF2. Panel d shows K_{1/2} values for the same complexes. Panel e shows an example of matched (MKI) and mismatched (MNI) dipeptidyl-RNCs with a sense codon (I) in the A site. Panel f shows rate constants for release (khyd) measured on several matched (MM, MKF, MFK) or mismatched (MNI, MNF, MLK) RNCs with saturating RF2. Rate constants for MM and MKF complexes are immeasurably low. Error bars indicate the standard error obtained from the nonlinear regression fit of the data.

FIG. 2 is a graph that shows abortive termination reaction is stimulated by the class II RF3 and is general for all P-site mismatches. Rate constants for release (k_{hyd}) for the indicated P-site-matched and mismatched (red) dipeptidyl-RNC complexes with RF2 only or with RF2 and RF3, all at saturating concentrations. fMet-Lys-tRNA^{Lys} occupies the P site in each case, either on cognate (AAA) or on first (UAA), second (AUA) or third (AAU) position mismatches. Error bars indicate the standard error obtained from the nonlinear regression fit of the data.

FIG. 3 (a and b) shows a single miscoding event promotes iterated errors in tRNA selection. a, is a graph that shows rate constants for peptidyl transfer (k_{PT}) for the indicated dipeptidyl-RNCs with either the cognate aminoacyl-tRNA (for MKI and MNI, total tRNA mix aminoacylated only with isoleucine, and for MKF and MNF, purified Phe-tRNA^{Phe}) or with bulk aminoacyl-tRNA. Error bars indicate the standard error obtained from the nonlinear regression fit of the data. b, shows two-dimensional TLCs resolve the peptidyl transfer reaction products resulting from reaction of the MM (left panel) or MNI (right panel) dipeptidyl-RNC with bulk aminoacylated tRNA; the reaction was incubated for 0.5 s, short of its endpoint. Mismatched base pairs are shown in red.

FIG. 4 (a-d) shows iterated miscoding results in doubly mismatched complexes, where release catalysis is dramatically promoted. a is a schematic representation of one of the tripeptidyl-RNC series used to address E-site effects on release; shown is the MKKF series. The MKKF complex carries no mismatches, MKNF contains a P-site mismatch, MNKF has an E-site mismatch, and MNNF bears both E- and P-site mismatches. Mismatched base pairs are shown in red. b-d are graphs showing rate constants for release (khyd) on a log scale with saturating RF2 and RF3 for the MKKF series (b), MEEP series (c) and MFFK series (d). Error bars indicate the standard error obtained from the nonlinear regression fit of the data.

FIG. 5 (a-c) shows an initial miscoding event results in an overall drop in yield of full-length peptides. a is a schematic showing a proposed model for the events after a miscoding event with the steps contributing to the quality control described here highlighted by green arrows. PT, peptidyl transfer. b, shows mock in vivo experiment recapitulates predictions of model. The indicated series of mRNAs (MKX to MKIFHKX for the matched series and MNX to MNIFHKX for the mismatched series) were used in complete translation reactions to observe the consequences of competition between tRNAs and release factors for peptide synthesis. Peptides initiated with the cognate dipeptide MK from the matched mRNA series are in black (MK-matched), peptides initiated with the cognate MN from the mismatched mRNA series are in blue (MN-matched), whereas peptides resulting from an incorrect decoding by Lys-tRNA^{Lys} on the Asn (N) codon are in red (MN-mismatched). c is a graph, where yield was quantified as the fractional radioactivity in each product band relative to the whole lane. The plot represents the average of three independent experiments, with error bars representing the standard deviation from the mean.

FIG. 6 (a and b) shows an estimate of misincoporation rate of in vitro translation mix. a is an autoradiograph of an electrophoretic TLC resolving the products of a 5 minute PT reaction between an initiation complex (with f-[35S]-Met-tRNA^{fMet} programmed in the P site with an mRNA coding for f-Met-Phe, AUG-UUC) and EF-Tu ternary complex containing the indicated aminoacylated tRNA (Phe and Leu indicate pure Phe-tRNA^{Phe} and Leu-tRNA₅^{Leu} tRNAs respectively, while mix indicates total E. coli tRNA purchased from Roche aminoacylated with full complement of twenty amino acids) in buffers A to D (Materials and Methods). PT reactions with Leu-tRNA^{Leu} yielded a secondary product that migrates to the same spot as the dipeptide fMet-Phe presumably due to contamination in the tRNA stock. Miscoding by tRNA^{Leu} is evidently the most common error in reactions containing aminoacyl-tRNA mixture. b, The error rate was estimated by quantifying the area beneath and above each of the reactions with the total tRNA relative to the corresponding area in the Phe only reaction. The quantitated value represents the average of two experiments.

FIG. 7 shows efficient decoding of the Asn codon AAU by Lys-tRNA^{Lys}. Dipeptidyl RNCs programmed with the indicated messages were prepared from ICs and were reacted with Lys-tRNA^{Lys}. The reaction mixture was resolved by electrophoretic TLC following hydrolysis with KOH.

FIG. 8 is a graph that shows rate of release is strongly affected by buffer condition and methylation status of RF2. Release rate constants for the dipepitdyl tRNA RNCs MKX and MYX (programmed with the mRNAs AUG-AAA-UGA and AUG-UAC-UGA, respectively) under the indicated conditions. Similar relative increases are observed with the two different complexes as a function of buffer conditions and the source of RF2, suggesting that these conditions have general effects on release that are not complex specific. (oe) indicates over-expressed RF2, while (ce) denotes a chromosomally-expressed (more heavily methylated) preparation.

FIG. 9 (a and b) shows RF2 releases a mismatched complex (MNF) with an efficiency that is >300 fold higher than the matched one (MKF). Panel a shows release time courses with the indicated RNCs were carried out at varying concentrations of RF2. Rates were determined, plotted against the enzyme concentration ([RF2]), and fit to a hyperbola (MNF) or a straight line (MKF). Error bars indicate the error associated with the fit for each time-course data. Panel b is a bar graph representing the measured second order rate constant (kcal Km) for the mismatched complex, or the inferred value (slope of the line) for the matched complex. Error bars are obtained from the non-linear regression in (a).

FIG. 10 shows the prescribed sense codon occupies the A site in the mismatched complex. Toe-printing experiment examined by PAGE for the MKI and MNI complexes. Initiation complexes (Init.) contain only f-Met-tRNA^{fMet} in the P site whereas elongated complexes (Elong.) are formed following the addition of Lys-tRNA^{Lys} ternary complex and EF-G and movement by 3 nts along the mRNA template. Red dots represent accurately initiated and elongated toeprint, with individual steps between shown with black dots.

FIG. 11 (a and b) shows release of mismatched complexes cannot be attributed to peptidyl-tRNA drop-off followed by peptidyl hydrolase-mediated hydrolysis. Panel a shows indicated complexes prepared with [32P]-labeled Lys-tRNA^{Lys} were incubated with excess deacylated, unlabeled tRNA^{Lys} and the off-rate of the dipeptidyl tRNA was followed as a function of time using a nitrocellulose filter-binding assay. Bars represent rates obtained from single exponential decay fits to the data with the error bars representing the error obtained from the non-linear regression of the data. The measured rates are much lower than those observed for RF2-mediated release. Panel b shows peptidyl hydrolase only catalyzes hydrolysis of the dipeptidyl tRNA in the sample that has been treated with EDTA to disassociate the ribosome complex (lane 5). Lack of hydrolysis of the dipeptidyl tRNA in the EDTA treated sample with no PTH indicates that there is no visible contamination of included components with PTH (lane 3).

FIG. 12 shows release of dipeptidyl tRNA from mismatched ribosome complex (MNF) is paromomycin sensitive. Autoradiograph of electrophoretic TLC demonstrating that paromomycin inhibits release of dipeptidyl tRNA from the mismatched RNC MNF.

FIG. 13 is a graph that shows wobble base-pairing is best tolerated at the third position of the P-site codon. K_{hyd} with saturating RF2 and RF3 for dipeptidyl-RNCs with either U:U or G:U (wobble) mispairings (mRNA:tRNA) in the P site at the 1st 2nd or 3rd codon positions. Error bars represent the error obtained from the non-linear regression of the data

FIG. 14 (a and b) shows two-dimensional TLC resolving the products of a PT reaction between the matched MKF (a) or mismatched MNF (b) complex with total aminoacylated tRNA.

FIG. 15 shows mismatched complex reacts more readily with near-cognate tRNAs. Autoradiograph of one-dimensional electrophoretic TLC resolving products of PT reaction between matched (MKF) and mismatched (MNF) RNCs and indicated pure aminoacylated tRNA (Phe, Val, Tyr, Leu). Note that Phe-tRNA^{Phe} is cognate for the two complexes (MKF and MNF) and the remaining three tRNAs are near cognate (1st 2nd and 3rd position).

FIG. 16 is a graph that shows relative promiscuous release activity on matched and mismatched RNCs is unaffected by buffer or RF2 source. Rate constants for the indicated complexes in buffer A with over-expressed RF2 (oe RF2), or in buffer D with over-expressed or chromosomally-expressed RF2 (ce RF2). Error bars represent the error obtained from the non-linear regression of the data

FIG. 17 (a and b) shows abortive termination is no longer triggered once errors have progressed out of the E site of the ribosome. Panel a is a schematic representation of tetrapetidyl RNCs used in this assay; cognate (MKKKF), out of the E site error (M(UAA)KKF), and P-site mismatch (MKK(UAA)F). Panel b is an autoradiograph of electrophoretic TLC showing that RF2/RF3 promotes premature release only on the P-site-mismatched complex but not the other two (reaction was carried out for 10 minutes).

FIG. 18 (a and b) shows homopolymeric complexes containing a mismatch in the E site undergo a frameshift. Toe-printing experiment examined by PAGE for the MKKF a, or MEEP b, series of complexes. Initiation complexes (Init.) contain only f-Met-tRNA^{fMet} in the P site, whereas elongated complexes (Elong.) result from the addition of ternary complex (Glu-tRNAGiu in a or Lys-tRNA^{Lys} in b) and EF-G. Note in this case, because of the expected addition of two amino acids, the anticipated shift in the toeprint is 6 nts (bottom red circle) relative to the initiation toe-print (top red circle).

FIG. 19 shows a complex containing mismatches in the P and E sites partitions equally between premature release or peptidyl transfer. The indicated tripeptidyl-tRNA RNCs, containing fMetLysLys-tRNA^{Lys} in the P site and Phe codon in the A site, were incubated with an 5100 extract containing 120 μM charged total tRNA for 5 minutes, and resolved by electrophoretic TLC.

FIG. 20 (a and b) shows a single miscoding event results in an overall drop in yield of full-length peptide in buffer D. Panel a shows mock in vivo experiment recapitulates predictions of model in buffer D. The indicated series of mRNAs (MKX through MKIFHKX for the matched series and MNX through MNIFHKX for the mismatched series) were used in complete translation reactions to observe the consequences of competition between tRNAs and RFs for peptide synthesis. Peptides initiated with the cognate dipeptide MK from the matched mRNA series are assigned the color black (MK-matched), peptides initiated with the cognate MN from the mismatched mRNA series are assigned the color blue (MN-matched), while peptides resulting from an incorrect decoding by Lys-tRNA^{Lys} on the Asn (N) codon are assigned the color red (MN-mismatched). Panel b is a graph that shows yield was quantified as the fractional radioactivity in each product band relative to the whole lane. Note that in buffer D, initial miscoding by Lys-tRNA^{Lys} is minimized relative to buffer A.

FIG. 21 shows protein yield is increased when overexpressed in a restrictive E. coli strain. The archaeal protein (minD-1) was overexpressed in the indicated strains, where WT, rpsL (rpsL141) and rpsD (rpsD12) indicate wild-type, hyper-accurate (restrictive) and error-prone (rain) strains, respectively. prfC::Kan indicates a strain where the gene encoding RF3 was deleted by replacing it with a Kanamycin-resistance gene, whereas pbadprfC indicates that RF3 was overexpressed. After overexpression of minD-1, total protein was separated using gel electrophoresis, and proteins of interest visualized by western-blotting with antigen-specific antibodies (α-His, α-RF2 and α-RF1). Note that ˜10 fold more of minD-1 is expressed in the rpsL strain relative to the WT one, under normal conditions (compare lane 1 to lane 4), while no expression was detected in the rpsD strain (lane 7). When RF3 was deleted, a modest increase in yield was observed in the WT strain. In the rpsD strain this increase was very significant (Lane 8). When RF3 was overexpressed, the levels of protein being produced dropped in all strains.

FIG. 22 shows deterioration of protein quality in an RF3-deletion strain. Glutathione S transferase (GST) was isolated from the indicated strains, WT and RF3-deletion (prfC::Kan) strains. The molecular weight of the protein was determined using MALDI-TOF (Mass spectrometry technique). When RF3 is deleted the peak corresponding to GST is widened indicating that in the absence of RF3, the GST protein is more heterogeneous. This heterogeneity is likely the result of uncorrected/undetected errors during protein synthesis because of the lack of post-PT quality control mechanism.

### DETAILED DESCRIPTION

The present inventors have identified a new form of quality control on the ribosome that results in the abortive termination of protein synthesis following the misincoporation of amino acids during protein synthesis. This discovery suggests a variety of approaches to manipulate the fidelity of the system—for example, through the removal or addition of translation components (e.g. release factors RF1, RF2 or RF3), through mutations in the ribosome, through drugs, and through tRNA abundance, to increase the yield of full length proteins.

The methods and compositions described here involve the use of a recombinant cell which is modified for overexpressing one or more release factors (RFs), so that protein expression is increased compared to a cell which has not been so modified. Such a recombinant cell may be produced using the methods described in further detail below.

The recombinant cell in certain preferred embodiments is prokaryotic, preferably an E. coli cell.

The recombinant cell in other preferred embodiments cell is a eukaryotic cell, preferably, but not limited to not limited to Saccharomyces cerevisiae, Pichia pastoris and Baculovirus cells.

The methods and compositions described herein also involve the use of a restrictive recombinant cell which is modified for overexpressing one or more release factors (RFs), so that protein expression is increased compared to a cell which has not been so modified. Such a restrictive recombinant cell may be produced using the methods described in further detail below.

The restrictive recombinant cell, in certain examples, is selected from, but not limited to, US157, UK285 and UK317.

The recombinant cell and recombinant restrictive cell may be used to produce any protein of interest, preferably at enhanced levels or higher yield, by comprising one or more release factors. Preferably, the release factor is selected from the group consisting of: release factor 1 (RF1), release factor 2 (RF2), and release factor 3 (RF3).

In certain examples, the release factor can be RF3.

In certain examples, the release factor can be RF2.

In certain examples, the release factor can be RF1.

In certain examples, the release factor can be RF2 and RF3

In certain examples, the release factor can be RF1 and RF3

In certain examples, the release factor can be RF1 and RF2

In certain examples, the release factor can be RF1, RF2 and RF3.

In further embodiments, RF1 is from E. coli and corresponds to SEQ ID NO: 1, shown below:

RF1 (E. coli):
SEQ ID NO: 1
MKPSIVAKLE ALHERHEEVQ ALLGDAQTIA DQERFRALSR
EYAQLSDVSR CFTDWQQVQE DIETAQMMLD DPEMREMAQD
ELREAKEKSE QLEQQLQVLL LPKDPDDERN AFLEVRAGTG
GDEAALFAGD LFRMYSRYAE ARRWRVEIMS ASEGEHGGYK
EIIAKISGDG VYGRLKFESG GHRVQRVPAT ESQGRIHTSA
CTVAVMPELP DAELPDINPA DLRIDTFRSS GAGGQHVNTT
DSAIRITHLP TGIVVECQDE RSQHKNKAKA LSVLGARIHA
AEMAKRQQAE ASTRRNLLGS GDRSDRNRTY NFPQGRVTDH
RINLTLYRLD EVMEGKLDML IEPIIQEHQA DQLAALSEQE

In other further embodiments, RF2 is from E. coli and corresponds to SEQ ID NO: 2, shown below:

RF2 (E. coli):
SEQ ID NO: 2
MFEINPVNNR IQDLTERSDV LRGYLDYDAK KERLEEVNAE
LEQPDVWNEP ERAQALGKER SSLEAVVDTL DQMKQGLEDV
SGLLELAVEA DDEETFNEAV AELDALEEKL AQLEFRRMFS
GEYDSADCYL DIQAGSGGTE AQDWASMLER MYLRWAESRG
FKTEIIEESE GEVAGIKSVT IKISGDYAYG WLRTETGVHR
LVRKSPFDSG GRRHTSFSSA FVYPEVDDDI DIEINPADLR
IDVYRTSGAG GQHVNRTESA VRITHIPTGI VTQCQNDRSQ
HKNKDQAMKQ MKAKLYELEM QKKNAEKQAM EDNKSDIGWG
SQIRSYVLDD SRIKDLRTGV ETRNTQAVLD GSLDQFIEAS
LKAGL

In still other further embodiments, RF3 is from E. coil and corresponds to SEQ ID NO: 3, shown below:

RF3 (E. coli):
SEQ ID NO: 3
MTLSPYLQEV AKRRTFAIIS HPDAGKTTIT EKVLLFGQAI
QTAGTVKGRG SNQHAKSDWM EMEKQRGISI TTSVMQFPYH
DCLVNLLDTP GHEDFSEDTY RTLTAVDCCL MVIDAAKGVE
DRTRKLMEVT RLRDTPILTF MNKLDRDIRD PMELLDEVEN
ELKIGCAPIT WPIGCGKLFK GVYHLYKDET YLYQSGKGHT
IQEVRIVKGL NNPDLDAAVG EDLAQQLRDE LELVKGASNE
FDKELFLAGE ITPVFFGTAL GNFGVDHMLD GLVEWAPAPM
PRQTDTRTVE ASEDKFTGFV FKIQANMDPK HRDRVAFMRV
VSGKYEKGMK LRQVRTAKDV VISDALTFMA GDRSHVEEAY
PGDILGLHNH GTIQIGDTFT QGEMMKFTGI PNFAPELFRR
IRLKDPLKQK QLLKGLVQLS EEGAVQVFRP ISNNDLIVGA
VGVLQFDVVV ARLKSEYNVE AVYESVNVAT ARWVECADAK
KFEEFKRKNE SQLALDGGDN LAYIATSMVN LRLAQERYPD
VQFHQTREH

The present invention provides generally for a number of RF1, RF2 and RF3 nucleic acids, together with fragments, homologues, variants and derivatives thereof. These nucleic acid sequences preferably encode the polypeptide sequences disclosed here, and particularly in the sequence listings.

Preferably, the polynucleotides comprise RF1, RF2 and RF3 nucleic acids. In certain embodiments, RF1, 3 and 3 are from E. coli and correspond to SEQ ID NO: 4, 5 and 6, respectively.

RF1 (E. coli) (ACCESSION CP001637 REGION:
2612384_2613464
SEQ ID NO: 4
ATGAAGCCTTCTATCGTTGCCAAACTGGAAGCCCTGCATGAACGCC
ATGAAGAAGTTCAGGCGTTGCTGGGTGACGCGCAAACTATCGCCGA
CCAGGAACGTTTTCGCGCATTATCACGCGAATATGCGCAGTTAAGT
GATGTTTCGCGCTGTTTTACCGACTGGCAACAGGTTCAGGAAGATA
TCGAAACCGCACAGATGATGCTCGATGATCCTGAAATGCGTGAGAT
GGCGCAGGATGAACTGCGCGAAGCTAAAGAAAAAAGCGAGCAACTG
GAACAGCAATTACAGGTTCTGTTACTGCCAAAAGATCCTGATGACG
AACGTAACGCCTTCCTCGAAGTCCGAGCCGGAACCGGCGGCGACGA
AGCGGCGCTGTTCGCGGGCGATCTGTTCCGTATGTACAGCCGTTAT
GCCGAAGCCCGCCGCTGGCGGGTAGAAATCATGAGCGCCAGCGAGG
GTGAACATGGTGGTTATAAAGAGATCATCGCCAAAATTAGCGGTGA
TGGTGTGTATGGTCGTCTGAAATTTGAATCCGGCGGTCATCGCGTG
CAACGTGTTCCTGCTACGGAATCGCAGGGTCGTATTCATACTTCTG
CTTGTACCGTTGCGGTAATGCCAGAACTGCCTGACGCAGAACTGCC
GGACATCAACCCAGCAGATTTACGCATTGATACTTTCCGCTCGTCA
GGGGCGGGTGGTCAGCACGTTAACACCACCGATTCGGCAATTCGTA
TTACTCACTTGCCGACCGGGATTGTTGTTGAATGTCAGGACGAACG
TTCACAACATAAAAACAAAGCTAAAGCACTTTCTGTTCTCGGTGCT
CGCATCCACGCTGCTGAAATGGCAAAACGCCAACAGGCCGAAGCGT
CTACCCGTCGTAACCTGCTGGGGAGTGGCGATCGCAGCGACCGTAA
CCGTACTTACAACTTCCCGCAGGGGCGCGTTACCGATCACCGCATC
AACCTGACGCTCTACCGCCTGGATGAAGTGATGGAAGGTAAGCTGG
ATATGCTGATTGAACCGATTATCCAGGAACATCAGGCCGACCAACT
GGCGGCGTTGTCCGAGCAGGAATAA
RF2 (E. coli) (ACCESSION CP001396 REGION:
2920354 . . . 2921452):
SEQ ID NO: 5
ATGTTTGAAATTAATCCGGTAAATAATCGCATTCAGGACCTCACGG
AACGCTCCGACGTTCTTAGGGGGTATCTTGACTACGACGCCAAGAA
AGAGCGTCTGGAAGAAGTAAACGCCGAGCTGGAACAGCCGGATGTC
TGGAACGAACCCGAACGCGCACAGGCGCTGGGTAAAGAGCGTTCCT
CCCTCGAAGCCGTTGTCGACACCCTCGACCAAATGAAACAGGGGCT
GGAAGATGTTTCTGGTCTGCTGGAACTGGCTGTAGAAGCTGACGAC
GAAGAAACCTTTAACGAAGCCGTTGCTGAACTCGACGCCCTGGAAG
AAAAACTGGCGCAGCTTGAGTTCCGCCGTATGTTCTCTGGCGAATA
TGACAGCGCCGACTGCTACCTCGATATTCAGGCGGGGTCTGGCGGT
ACGGAAGCACAGGACTGGGCGAGCATGCTTGAGCGTATGTATCTGC
GCTGGGCAGAATCGCGTGGTTTCAAAACTGAAATCATCGAAGAGTC
GGAAGGTGAAGTGGCGGGTATTAAATCCGTGACGATCAAAATCTCC
GGCGATTACGCTTACGGCTGGCTGCGTACAGAAACCGGCGTTCACC
GCCTGGTGCGTAAAAGCCCGTTTGACTCCGGCGGTCGTCGCCACAC
GTCGTTCAGCTCCGCGTTTGTTTATCCGGAAGTTGATGATGATATT
GATATCGAAATCAACCCGGCGGATCTGCGCATTGACGTTTATCGCA
CGTCCGGCGCGGGCGGTCAGCACGTTAACCGTACCGAATCTGCGGT
GCGTATTACCCACATCCCGACCGGGATCGTGACCCAGTGCCAGAAC
GACCGTTCCCAGCACAAGAACAAAGATCAGGCCATGAAGCAGATGA
AAGCGAAGCTTTATGAACTGGAGATGCAGAAGAAAAATGCCGAGAA
ACAGGCGATGGAAGATAACAAATCCGACATCGGCTGGGGCAGCCAG
ATTCGTTCTTATGTCCTTGATGACTCCCGCATTAAAGATCTGCGCA
CCGGGGTAGAAACCCGCAACACGCAGGCCGTGCTGGACGGCAGCCT
GGATCAATTTATCGAAGCAAGTTTGAAAGCAGGGTTATGA
RF3 (E. coli) (ACCESSION CP001637 REGION:
3902026 . . . 3903615):
SEQ ID NO: 6
ATGACGTTGTCTCCTTATTTGCAAGAGGTGGCGAAGCGCCGCACTT
TTGCCATTATTTCTCACCCGGACGCCGGTAAGACTACCATCACCGA
GAAGGTGCTGCTGTTCGGACAGGCCATTCAGACCGCCGGTACAGTA
AAAGGCCGTGGTTCCAACCAGCACGCTAAGTCGGACTGGATGGAGA
TGGAAAAGCAGCGTGGGATCTCCATTACTACGTCTGTGATGCAGTT
TCCGTATCACGATTGCCTGGTTAACCTGCTCGACACCCCGGGGCAC
GAAGACTTCTCGGAAGATACCTATCGTACCCTGACGGCGGTGGACT
GCTGCCTGATGGTTATCGACGCCGCAAAAGGTGTTGAAGATCGTAC
CCGTAAGCTGATGGAAGTTACCCGTCTGCGCGACACGCCGATCCTC
ACCTTTATGAACAAACTTGACCGTGATATCCGCGACCCGATGGAGC
TGCTCGATGAAGTTGAGAACGAGCTGAAAATCGGCTGTGCGCCGAT
CACCTGGCCGATTGGCTGCGGCAAGCTGTTTAAAGGCGTTTACCAC
CTTTATAAAGACGAAACCTATCTCTATCAGAGCGGTAAAGGCCACA
CCATTCAGGAAGTCCGCATTGTTAAAGGGCTGAATAACCCGGATCT
CGATGCTGCGGTTGGTGAAGATCTGGCACAGCAGCTGCGTGACGAA
CTGGAACTGGTGAAAGGCGCGTCTAACGAGTTCGACAAAGAGCTGT
TCCTTGCGGGCGAAATCACTCCGGTATTCTTCGGTACTGCGCTGGG
TAACTTCGGCGTCGATCATATGTTGGATGGCCTGGTGGAGTGGGCA
CCTGCGCCGATGCCGCGTCAGACTGATACCCGTACCGTAGAAGCGA
GCGAAGATAAATTTACCGGCTTCGTATTTAAAATTCAGGCCAACAT
GGACCCGAAACACCGCGACCGCGTGGCGTTTATGCGTGTGGTGTCC
GGTAAATATGAAAAAGGCATGAAACTGCGCCAGGTGCGCACTGCGA
AAGATGTGGTGATCTCCGACGCGCTGACCTTTATGGCGGGTGACCG
TTCGCACGTTGAAGAAGCGTATCCGGGCGATATCCTCGGCCTGCAC
AACCACGGCACCATTCAGATCGGCGACACCTTTACCCAGGGTGAGA
TGATGAAGTTCACCGGTATTCCGAACTTCGCACCAGAACTGTTCCG
TCGTATCCGCCTGAAAGATCCGCTGAAGCAAAAACAGCTGCTCAAA
GGGCTGGTACAGCTTTCCGAAGAGGGCGCGGTGCAGGTGTTCCGTC
CAATCTCCAACAACGATCTGATCGTTGGTGCAGTTGGTGTGCTGCA
GTTTGATGTGGTGGTAGCGCGCCTGAAGAGCGAATACAACGTTGAA
GCAGTGTATGAGTCAGTCAACGTTGCCACTGCCCGCTGGGTAGAAT
GTGCAGACGCGAAGAAATTCGAAGAGTTCAAGCGTAAGAACGAAAG
CCAACTGGCGCTTGATGGCGGCGATAACCTCGCTTACATCGCTACC
AGCATGGTCAACCTGCGCCTGGCACAGGAACGTTATCCGGACGTTC
AGTTCCACCAGACCCGCGAGCATTAA

In particular, the present invention provides for nucleic acids or polynucleotides which encode any of the RF1, RF2 or RF3 polypeptides disclosed here. Preferably, however, such nucleic acids or polynucleotides comprise any of the sequences set out as SEQ ID NOs: 4, 5 or 6, or a sequence encoding any of the corresponding polypeptides, and a fragment, homologue, variant or derivative of such a nucleic acid. The above terms therefore preferably should be taken to refer to these sequences.

As used herein, the terms “polynucleotide”, “nucleotide”, and nucleic acid are intended to be synonymous with each other. “Polynucleotide” generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

The terms “variant”, “homologue” or “derivative” in relation to a nucleotide sequence include any substitution of, variation of, modification of, replacement of, deletion of or addition of one (or more) nucleotides from or to the sequence. Preferably, the resulting sequence is capable of encoding a polypeptide which has apoptosis mediator activity.

As indicated above, with respect to sequence identity, a “homologue” has preferably at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to the relevant sequence shown in the sequence listings.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

The polypeptides disclosed include homologous sequences obtained from any source, for example related viral/bacterial proteins, cellular homologues and synthetic peptides, as well as variants or derivatives thereof. Thus polypeptides also include those encoding homologues of RF1, RF2 or RF3 from other species including animals such as mammals (e.g. mice, rats or rabbits), in particular rodents.

In the context of the present document, a homologous sequence or homologue is taken to include an amino acid sequence which is at least 60, 70, 80 or 90% identical, preferably at least 95 or 98% identical at the amino acid level over at least 30, preferably 50, 70, 90 or 100 amino acids with RF1, RF2 or RF3, as the case may be, for example as shown in the sequence listing herein. In the context of this document, a homologous sequence is taken to include an amino acid sequence which is at least 15, 20, 25, 30, 40, 50, 60, 70, 80 or 90% identical, preferably at least 95 or 98% identical at the amino acid level, preferably over at least 15, 25, 35, 50 or 100, preferably 200, 300, 400 or 500 amino acids with the sequence of RF1, RF2 and RF3.

In preferred embodiments, a RF1, RF2 or RF3 polypeptide has at least 98.1% or more sequence identity to a sequence shown as SEQ ID NO: 1, 2 or 3. Preferably, the RF1, RF2 or RF3 polypeptide has 98.2% or more, preferably 98.3% or more, 98.4% or more, 98.5% or more, 98.6% or more, 98.7% or more, 98.8% or more, 98.9% or more, 99.0% or more or 99.1% or more, 99.2% or more, preferably 99.3% or more, 99.4% or more, 99.5% or more, 99.6% or more, 99.7% or more, 99.8% or more, 99.9% or more sequence identity to a sequence shown as SEQ ID NO: 1, 2 or 3.

Although homology can also be considered in terms of similarity (i.e. amino acid residues having similar chemical properties/functions), in the context of the present document it is preferred to express homology in terms of sequence identity. In highly preferred embodiments, the sequence identity is determined relative to the entirety of the length the relevant sequence, i.e., over the entire length or full length sequence of the relevant gene, for example.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximize local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The protein of interest may include those that are of therapeutic and/or diagnostic application such as, but not limited to: sequences encoding cytokines, chemokines, hormones, antibodies, anti-oxidant molecules, engineered immunoglobulin-like molecules, a single chain antibody, a humanized antibody, fusion proteins, enzymes, a toxin, a conditional toxin, an antigen, a tumor suppresser protein and growth factors, membrane proteins, vasoactive proteins and peptides, anti-viral proteins and enzymes, and derivatives thereof (such as with an associated reporter group), pro-drug activating enzymes or proteins used for academic research.

Certain genes can provide challenges for efficient expression by recombinant means in heterologous hosts. Alteration of the codons native to the sequence can facilitate more robust expression of these proteins. Codon preferences for abundantly expressed proteins have been determined in a number of species, and can provide guidelines for codon substitution. Synthesis of codon-optimized sequences can be achieved by substitution of codons in cloned sequences, e.g., by site-directed mutagenesis, or by construction of oligonucleotides corresponding to the optimized sequence by chemical synthesis. See, e.g., Mirzabekov et al., J. Biol. Chem., 274:28745-50, 1999.

The optimization should also include consideration of other factors such as the efficiency with which the sequence can be synthesized in vitro (e.g., as oligonucleotide segments) and the presence of other features that affect expression of the nucleic acid in a cell. For example, sequences that result in RNAs predicted to have a high degree of secondary structure should be avoided. AT- and GC-rich sequences that interfere with DNA synthesis should also be avoided. Other motifs that can be detrimental to expression include internal TATA boxes, chi-sites, ribosomal entry sites, prokaryotic inhibitory motifs, cryptic splice donor and acceptor sites, and branch points. These features can be identified manually or by computer software and they can be excluded from the optimized sequences.

Accordingly, in certain preferred embodiments, the recombinant cell further comprises a vector comprising a codon optimized gene encoding the protein.

The present inventors have identified a new form of quality control on the ribosome that results in the abortive termination of protein synthesis following the misincoporation of amino acids during protein synthesis. It is an object of the current invention to increase protein expression.

Accordingly, in the recombinant and restrictive recombinant cells described herein, post-peptidyl transfer quality control is increased.

In other embodiments, in the recombinant and restrictive recombinant cells described herein, the fidelity of tRNA selection is increased.

In other preferred embodiments, in the recombinant and restrictive recombinant cells described herein, the rate of incorporation of amino acids is suppressed.

### Methods

The present invention features methods for producing recombinant proteins.

In certain embodiments, the methods comprise culturing a recombinant cell or recombinant restrictive cell comprising one or more release factors, as described herein, under conditions such that one or more proteins are overexpressed, and isolating the expressed recombinant protein. Preferably, the methods of the invention further comprise a step of transfecting or transforming the cell with a gene encoding the protein.

In certain preferred embodiments, the gene encoding the protein is codon optimized.

In certain examples, a vector may be used to replicate the nucleic acid in a compatible host cell. Accordingly, the vector can be introduced into a compatible host cell, and the host cell is grown under conditions which bring about replication of the vector. The vector may be recovered from the host cell. Suitable host cells include bacteria such as E. coli, yeast, mammalian cells and other eukaryotic cells.

A polynucleotide in a vector is preferably operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

The nucleotide vectors, preferably expression vectors, may suitably comprise coding sequences for any protein of interest which it is desired to express. Alternatively, or in addition, the protein of interest may be expressed from a separate expression vector, which may be constructed in an analogous fashion to the methods described here.

Vectors may be transformed or transfected into a suitable host cell as described below to provide for expression of a protein. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the protein include promoters/enhancers and other expression regulation signals. These control sequences may be selected to be compatible with the host cell for which the expression vector is designed to be used in. The term “promoter” is well-known in the art and encompasses nucleic acid regions ranging in size and complexity from minimal promoters to promoters including upstream elements and enhancers.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter, the Rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

In order to express a biologically active polypeptide, a polynucleotide sequence of interest is brought into association with a regulatory sequence so as to enable the regulatory sequence to direct expression of said polynucleotide. Expression of the polypeptide under control of the regulatory sequence is then allowed to happen. Optionally, the polypeptide so produced may be purified. Preferably, the regulatory sequence is one with which the polynucleotide sequence is not naturally associated.

The nucleotide sequences encoding the respective nucleic acid or homologues, variants, or derivatives thereof may be inserted into appropriate expression vector, i.e., a vector which contains the necessary elements for the transcription and translation of the inserted coding sequence.

Methods of enabling expression of polypeptides are set out below.

One method by which to provide expressed polypeptides is by means of an expression vector, i.e., a vector (e.g., a plasmid) which contains a regulatable promoter, optionally with other regulatory sequences such as enhancers, which is operably linked to a sequence encoding a polypeptide of interest which has been cloned into the expression vector.

Methods which are well known to those skilled in the art may be used to construct expression vectors containing sequences encoding the protein of interest and appropriate transcriptional and translational control elements. These methods include in vitro recombinant DNA techniques, synthetic techniques, and in vivo genetic recombination. Such techniques are described in Sambrook, J. et al. (1989; Molecular Cloning, A Laboratory Manual, ch. 4, 8, and 16-17, Cold Spring Harbor Press, Plainview, N.Y.) and Ausubel, F. M. et al. (1995 and periodic supplements; Current Protocols in Molecular Biology, ch. 9, 13, and 16, John Wiley & Sons, New York, N.Y.).

A variety of expression vector/host systems may be utilized, and include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. Any suitable host cell may be employed.

The “control elements” or “regulatory sequences” are those non-translated regions of the vector (i.e., enhancers, promoters, and 5′ and 3′ untranslated regions) which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, La Jolla, Calif.) or PSPORT1 plasmid (GIBCO/BRL), and the like, may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector.

In mammalian cell systems, promoters from mammalian genes or from mammalian viruses are preferable.

In bacterial systems, a number of expression vectors may be selected depending upon the use intended, including, but not limited to, multifunctional E. coli cloning and expression vectors such as BLUESCRIPT (Stratagene).

In the yeast Saccharomyces cerevisiae, a number of vectors containing constitutive or inducible promoters, such as alpha factor, alcohol oxidase, and PGH, may be used. For reviews, see Ausubel (supra) and Grant et al. (1987; Methods Enzymol. 153:516-544).

In mammalian host cells, a number of viral-based expression systems may be utilized. In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, and W138), are available from the American Type Culture Collection (ATCC, Bethesda, Md.) and may be chosen to ensure the correct modification and processing of the foreign protein.

For long term, high yield production of recombinant proteins, stable expression is preferred. For example, cells capable of stably expressing a protein of interest can be transformed using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cells. These include, but are not limited to, the herpes simplex virus thymidine kinase genes (Wigler, M. et al. (1977) Cell 11:223-32) and adenine phosphoribosyltransferase genes (Lowy, I. et al. (1980) Cell 22:817-23), which can be employed in tK- or apr-cells, respectively. Also, antimetabolite, antibiotic, or herbicide resistance can be used as the basis for selection. For example, dhfr confers resistance to methotrexate (Wigler, M. et al. (1980) Proc. Natl. Acad. Sci. 77:3567-70); npt confers resistance to the aminoglycosides neomycin and G418 (Colbere-Garapin, F. et al (1981) J. Mol. Biol. 150:1-14); and als or pat confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine. (Hartman, S. C. and R. C. Mulligan (1988) Proc. Natl. Acad. Sci. 85:8047-51.) Recently, the use of visible markers has gained popularity with such markers as anthocyanins, .beta.-glucuronidase and its substrate GUS, and luciferase and its substrate luciferin. These markers can be used not only to identify transformants, but also to quantify the amount of transient or stable protein expression attributable to a specific vector system. (Rhodes, C. A. et al. (1995) Methods Mol. Biol. 55:121-131.)

Although the presence/absence of marker gene expression suggests that the gene of interest is also present, the presence and expression of the gene may need to be confirmed. For example, if the sequence encoding the protein of interest is inserted within a marker gene sequence, transformed cells containing sequences encoding the protein of interest can be identified by the absence of marker gene function. Alternatively, a marker gene can be placed in tandem with a sequence encoding the protein of interest under the control of a single promoter. Expression of the marker gene in response to induction or selection usually indicates expression of the tandem gene as well.

Alternatively, host cells which contain the nucleic acid sequence encoding a protein of interest may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein sequences.

A variety of protocols for detecting and measuring the expression of the protein of interest, using either polyclonal or monoclonal antibodies specific for the protein, are known in the art. Examples of such techniques include enzyme-linked immunosorbent assays (ELISAs), radioimmunoassays (RIAs), and fluorescence activated cell sorting (FACS). These and other assays are well described in the art, for example, in Hampton, R. et al. (1990; Serological Methods, a Laboratory Manual, Section IV, APS Press, St Paul, Minn.) and in Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding a protein or proteins of interest include oligo labeling, nick translation, end-labeling, or PCR amplification using a labeled nucleotide. Alternatively, said sequences, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding a protein of interest may be cultured under conditions suitable for the expression and recovery of the protein from cell culture. The protein produced by a transformed cell may be located in the cell membrane, secreted or contained intracellularly depending on the sequence and/or the vector used. As will be understood by those of skill in the art, expression vectors containing polynucleotides which encode a protein or proteins of interest may be designed to contain signal sequences which direct secretion through a prokaryotic or eukaryotic cell membrane. Other constructions may be used to join sequences encoding the protein of interest to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). A discussion of vectors which contain fusion proteins is provided in Kroll, D. J. et al. (1993; DNA Cell Biol. 12:441-453).

Fragments of polypeptides, as well as whole length polypeptides, may be produced not only by recombinant production, but also by direct peptide synthesis using solid-phase techniques. (Merrifield J. (1963) J. Am. Chem. Soc. 85:2149-2154.) Protein synthesis may be performed by manual techniques or by automation. Automated synthesis may be achieved, for example, using the Applied Biosystems 431A peptide synthesizer (Perkin Elmer). Various fragments may be synthesized separately and then combined to produce the full length molecule.

Accordingly, the invention features a recombinant protein produced by any of the methods described herein.

The expression yield of the protein is higher compared to cells which do not comprise one or more release factors as described herein.

For example, the expression yield may be at least 1.1 times. than of cells which have not been so modified. In further preferred embodiments, the expression yield may be at least 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 10 times or more than of cells which do not comprise one or more release factors.

In some embodiments, the recombinant or recombinant restrictive cell is cultured in a suspension culture.

In some embodiments, the recombinant or recombinant restrictive cell is cultured in a batch culture. By batch culture is meant to refer to a closed system culture of microorganisms where a limited number of generations are allowed to grow before all nutrients are used up. Such a closed system may contain conditions to enable cell culture growth, such as specific nutrient types, temperature, pressure, aeration, and other environmental conditions.

In other embodiments, the recombinant or recombinant restrictive cell is cultured in a fed-batch culture. A fed-batch culture is understood to mean a method of culturing cells in which a solution of nutrients is added at intervals during culture. In fed-batch mode, usually little or no product is harvested until the end of the run.

In some embodiments, the recombinant or recombinant restrictive cell is cultured in a bioreactor.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

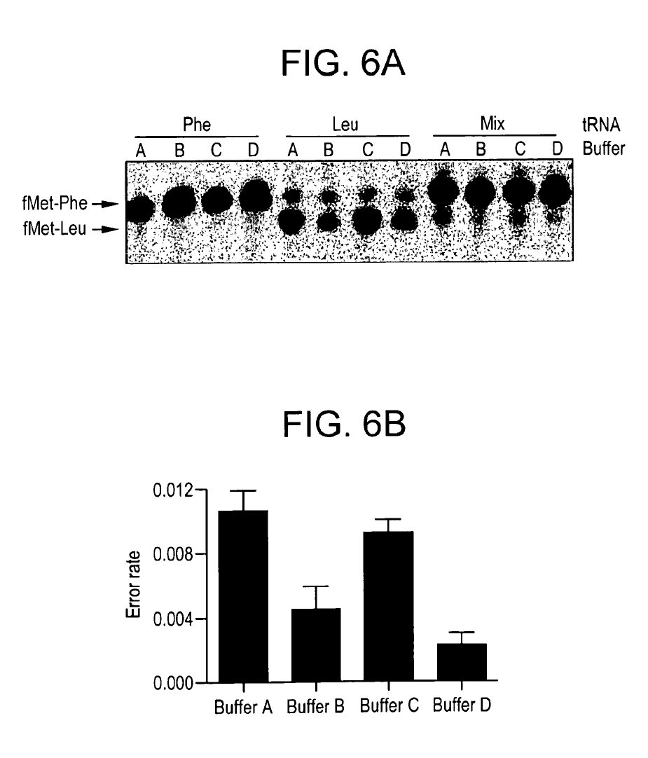

The overall in vivo rate of misincoporation during protein synthesis has been estimated to be in the range of 6×10⁴ to 5×10³ per amino acid incorporated (1,2). Current models for the mechanisms governing this level of accuracy focus on the accurate charging of tRNAs with their cognate amino acid by the aminoacyl-tRNA synthetases and correct tRNA selection by the ribosome facilitated by the GTPase elongation factor EF-Tu in bacteria (or EF1A in eukaryotes). Kinetic discrimination mechanisms, driven by induced fit, have been demonstrated for the synthetases and the ribosome to facilitate accurate selection of amino adds or charged tRNAs, respectively (3,4). In addition, for both processes, proofreading (or editing) mechanisms have been shown to increase the overall fidelity further (3, 5-7). Experimental measurements of in vitro aminoacylation accuracy (˜10⁵) agree well with that observed in vivo (8). In vitro protein synthesis systems (generally poly-Phe synthesis on polyU) have been shown to proceed with an overall fidelity (combining the tRNA selection and proofreading steps) of as high as 10⁻⁴ (refs 3, 9 and 10). However, fidelity measurements conducted by the present inventors in the full range of published buffer systems with tRNA mixtures on heteropolymeric messenger RNA suggest that in vitro protein synthesis proceeds with slower fidelity (an error rate of 2×10⁻³ to 10×10⁻³; FIG. 6), thus demonstrating that further quality control mechanisms may exist.

The present experiments identify a previously uncharacterized ribosome-centered mechanism that contributes to translational quality control, and which may help explain discrepancies between in vitro and in vivo measured fidelity values. The surprising feature of this pathway is that it monitors the fidelity of protein synthesis after the formation of a peptide bond (retrospectively), in certain ways analogous to the exonucleolytic proofreading step in DNA replication (11). The present data provides evidence that the ribosome recognizes errors during synthesis by evaluating the codon—anticodon helix in the peptidyl (P) site of the small subunit of the ribosome, leading first to reduced fidelity during subsequent tRNA selection and ultimately to premature termination by release factors.

### Example 1

### A Mismatched P Site Triggers Unusual Release Behavior

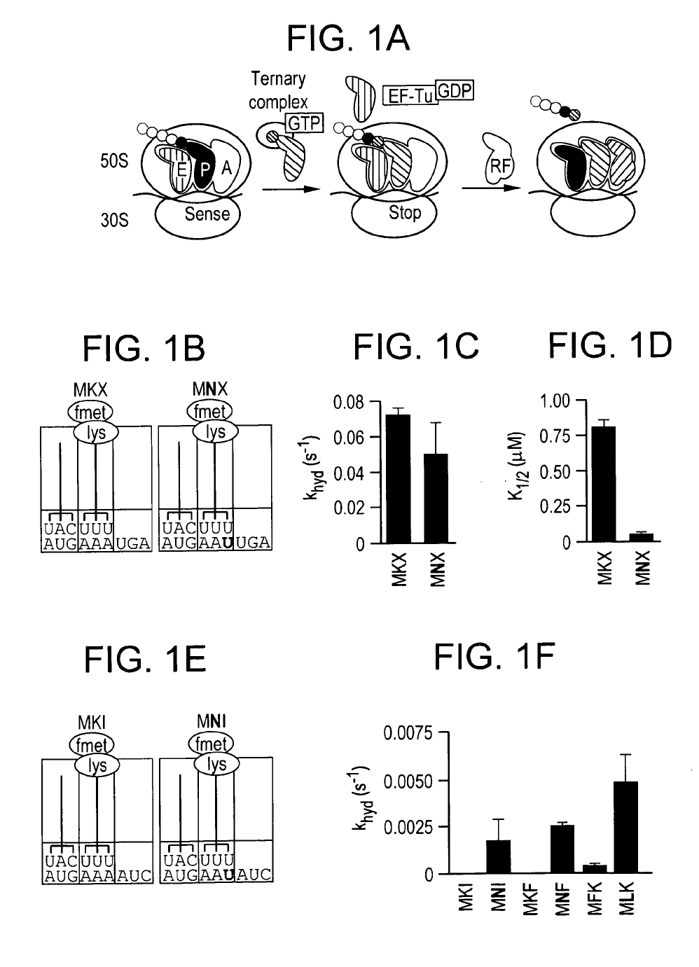

During the course of reconstituting in vitro the translation of ribosome nascent chain complexes (RNCs), with the cure elongation steps shown in FIG. 1a, an abundant miscoding event was identified in which Lys-tRNA (anticodon UUU) efficiently decoded an AAU Asu codon in a short peptide sequence, as previously documented in vivo (12). In these reactions, it was observed that in miscoded ribosome complexes the peptidyl-tRNA did not efficiently react to incorporate the next amino acid encoded by the mRNA, but instead seemed to be promiscuously hydrolyzed. These data suggested the existence of a quality control step that follows peptide bond formation and effectively functions to terminate translation of aberrant protein products, thus enhancing the overall fidelity of protein synthesis.

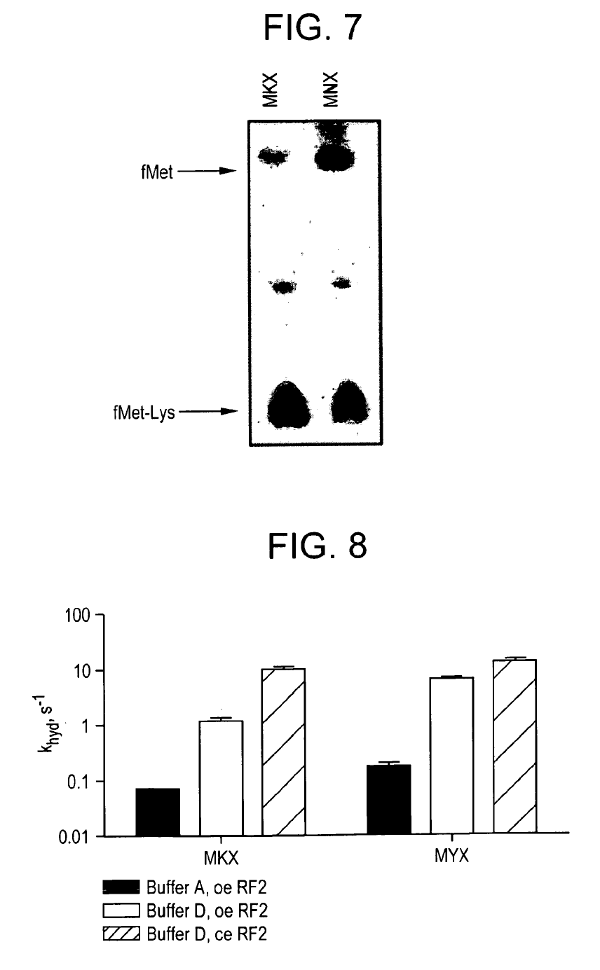

To characterize this unusual observation, RNCs were produced carrying a dipeptidyl-tRNA in the P site with either a matched or a mismatched codon-anticodon helix and a variety of different codons in the A site. A pair of RNCs containing a stop codon in the A site (mRNAs encoding MKX (AUG AAA UGA) or MNX (AUG AAU UGA)) were used to check anticipated release factor 2 (RF2) properties on these complexes. Complexes were assembled in a simplified reaction mixture containing initiation factors (IF1-IF3) and fMet-tRNA^{fMet}, and were reacted with ternary complex (Lys-tRNA^{Lys}-EF-Tu-GTP) in the presence of elongation factor EF-G to yield ribosome complexes with fMet-Lys-tRNA^{Lys} in the P site (FIG. 1b and FIG. 7), followed by purification over a sucrose cushion. As anticipated, RF2 reacted efficiently with both complexes (MKX and MNX), releasing the dipeptide with a rate constant (kᵣₐₜ) dose to that previously reported in buffer A (−0.05 s−1, FIG. 1c) Notably, titration experiments indicated that less RF2 was required to promote the maximal rate of catalysis on the mismatched P site complex than on the matched one (K_{1/2}, release factor concentration at which half of the maximal rate is observed, values of 75 nM and 800 nM, respectively, FIG. 1d). These data suggested that RF2 interacts differently with these two complexes that vary by a single mismatch in the P site. The maximal rate of release on other matched stop-codon-programmed complexes was, as expected, dependent on the buffer used and on the source of RF2 (over- or chromosomally-expressed) reaching a maximum of 10 s−1, close to numbers reported previously (15) (FIG. 8). Although maximal rates of release are achieved in buffer D, the study was completed in buffer A because background release rates were minimal under these conditions

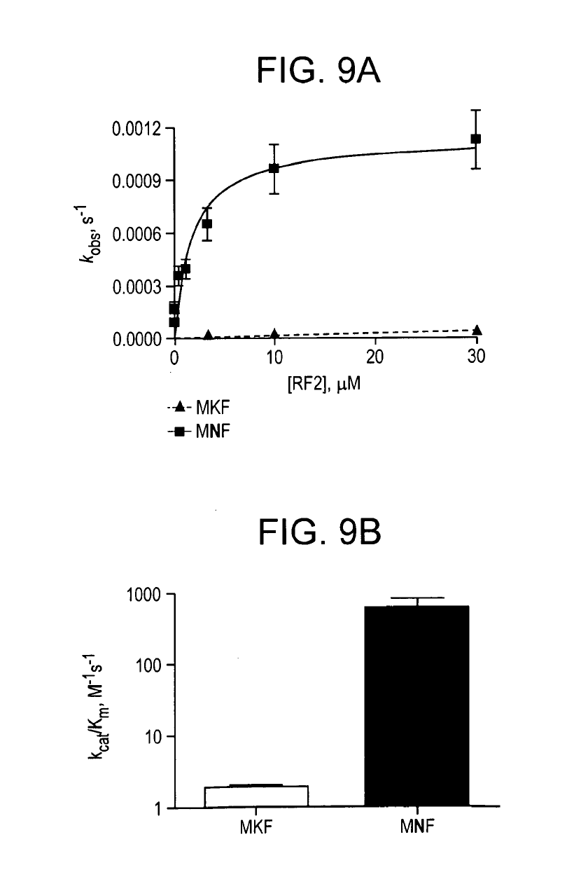

Next, several dipeptidyl-tRNA ribosomal complexes that instead carried sense codons in the A site were prepared and their behavior was assessed in pre-steady state release assays. In the first set, complexes MKI and MNI carrying tMet-Lys-tRNA^{Lys} in the P site and the Ile codon AUC in the A site were used (FIG. 1e). RF2-catalyzed release on the sense codon was immeasurably slow for the matched (MKT) complex (kₐₜ<0.0002 s−1), as expected (16), but was markedly faster (k_{cat} ˜0.002 s−1) for the P-site-mismatched complex (FIG. 1f). Next, similar matched and mismatched ribosome complexes, MKF and MNF, were prepared, carrying a different A-site codon (Phe, UUU). As before, the k_{cat} of the mismatched P-site complex (MNF) was markedly faster than that of the matched complex (MKF; 0.0025 s⁻¹ versus <0.0001 s⁻¹, respectively, FIG. 1f. The second order rate difference (k_{cat}/Kₘ) was considerably greater for the mismatched complex MNF relative to MKF (>300-fold, FIG. 9).

To determine whether the increased reactivity of the P-site mismatched complex depended on particular decoding mistakes or if the phenomenon was more general, a different near-cognate pairing interaction was forced in order to prepare matched and mismatched dipeptidyl tRNA complexes (MFK and MLK). These complexes contained several differences compared to the previous set (P-site tRNA, P-site mismatch and A-site codon). Again, RF2-catalyzed peptide release was considerably faster for the P-site-mismatched complex than for the matched version (0.0048 s−1 versus 0.0004 s−′, respectively, FIG. 1f). The related release factor, RF1, showed similar promiscuous release activity (data not shown), but was not be further characterized in this study.

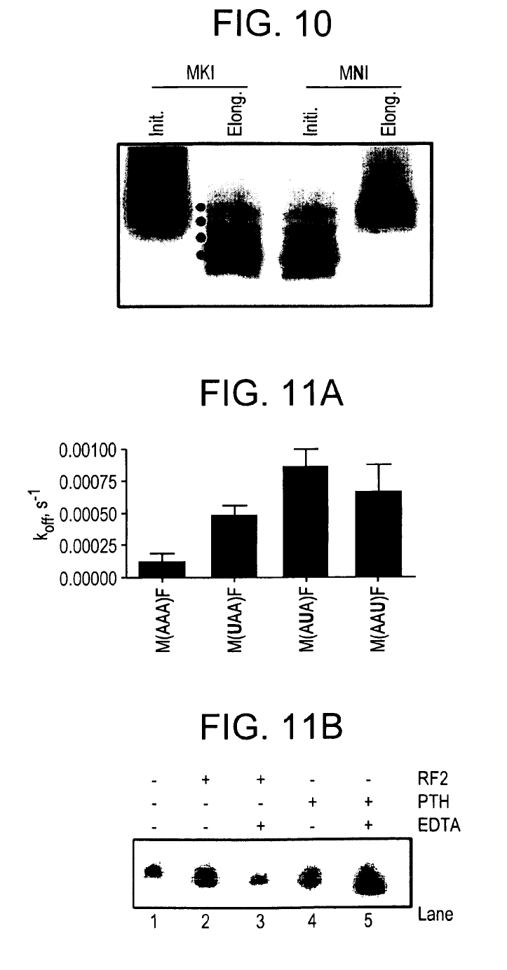

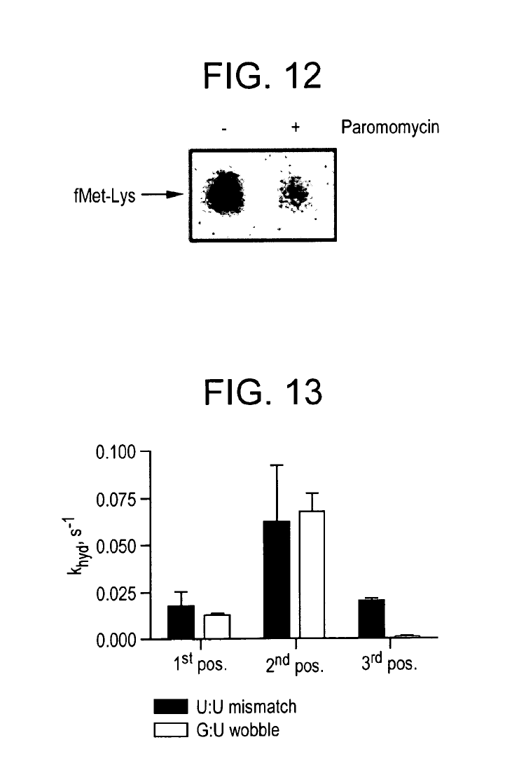

Given the apparent generality of the observed release-factor promiscuity on sense codons, several experiments were performed to determine whether this phenomenon is an authentic ribosome-based event. One concern was that the mismatch in the P site might destabilize the complex and permit a frame shifting or hopping event on the mRNA. that might reposition au authentic stop ((Aim in the A site. Although the mRNAs were designed to avoid this potential complication, the positioning of the ribosome on the mRNA in the matched and mismatched ribosome complexes was examined using a primer-extension based toe-printing assay (18). The primer extension reactions on MKI and MNI have indistinguishable toe-printing patterns, consistent with a 3-nucleotide shift of the complex after a round of elongation (FIG. 10). Further controls established that hydrolysis cannot be attributed to P-site tRNA drop-off followed by hydrolysis by potential contaminating peptidyl hydrolase (FIG. 11). Furthermore, the promiscuous release activity, like authentic release, was inhibited by paromomycin (14, 19). (FIG. 12). These experiments together show that the observed activity on the P-site-mismatched complexes reports on an authentic release-factor-mediated ribosomal event.

### Example 2

### RF3 Stimulates Promiscuous Release by RF2

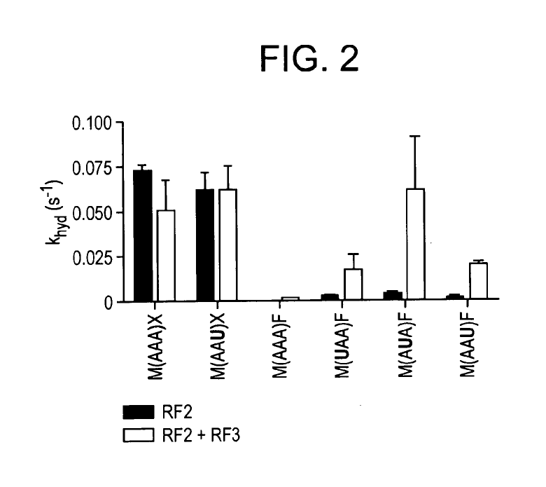

Although the observed stimulation of premature peptide release on the sense codons of P-site-mismatched complexes was substantial (>20-fold), the resulting rate constant for the reaction (0.002 [symbol] 0.005 s⁻¹) still lagged behind that of authentic peptide release (˜0.05 s⁻¹) and the competing tRNA selection/peptidyl transfer processes (˜2 s⁻¹). Class II release factor RF3 is a GTPase that is integrally involved in the removal of the class I release factor after peptide release, but has no effect on the rate constants for peptide release on authentic termination complexes (FIG. 2 and ref. 22). Interestingly, when RF2 and RF3 were added together to a variety of P-site-mismatched complexes (with first, second and third position mismatches in the P site), release activity was substantially accelerated (˜20-50-fold, FIG. 2). It was noted that the resulting rate constants for the release reaction can be remarkably fast for some complexes (˜0.1 s⁻¹), in a range in which this promiscuity could influence the fidelity of protein synthesis in vivo. Notably, it has been previously shown that RF3 could stimulate release on certain ribosome complexes containing a near-cognate stop codon in the A site (16).

A related question is whether the mechanism that monitors miscoding is sensitive to the identity of the mismatch found at each of the three positions of the codon. The U•U mismatch and U•G wobble resulted in similar release activity when located at the first or second codon positions; in contrast, at the third codon position, whereas the U•U mismatch strongly stimulated peptide release, the U•G wobble had little consequence (FIG. 13). These data are hilly consistent with expectations on the basis of the permissivity of the genetic code at the third co dun position and argue that the system fur monitoring fidelity in the P site can identify a wide range of errors during translation.

### Example 3

### P-Site Mismatches Compromise tRNA Selection Fidelity

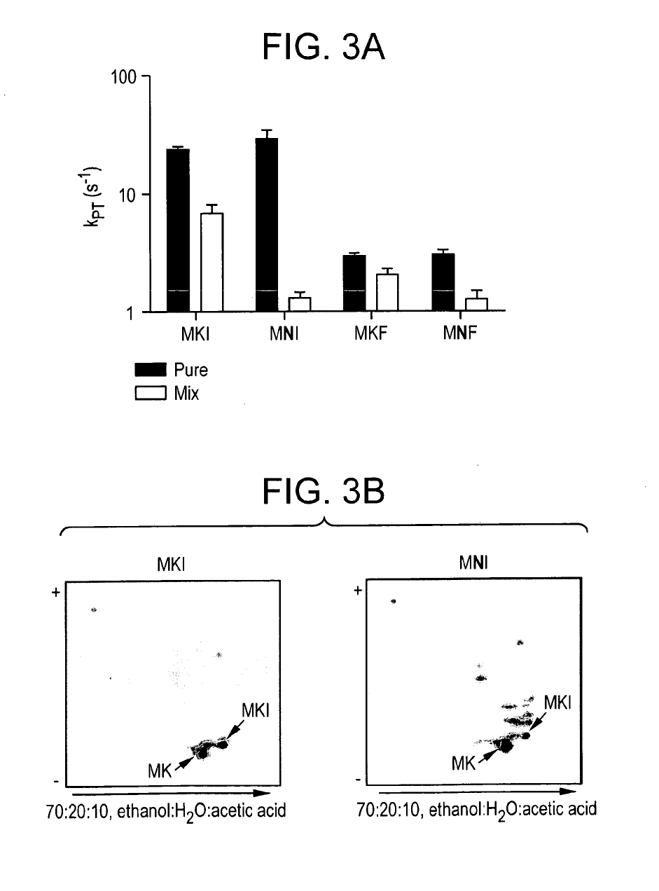

Class I release factors must naturally compete during translation with the cognate and near-cognate tRNA species that sample the same ribosomal A site. Given the apparent substantial effects of the P-site mismatch on release factor activity, next the peptidyl transfer activity of the P-site-matched and mismatched complexes was examined. In two different examples (MKI versus MNI, and MKF versus MNF), it was observed that the rate constant for peptidyl transfer (for cognate Ile-tRNA^{Ile} and Phe-tRNA^{Phe}, respectively) was unaffected by the mismatch in the P site (FIG. 3a). It was observed, however, that catalysis of peptidyl transfer is diminished by twofold to sixfold for the P-site-mismatched complexes relative to the matched complexes in the presence of a full complement of competitor tRNA (FIG. 3a). It is proposed that this reduced overall peptidyl transfer rate results from the increased dwell time of near-cognate tRNAs on the P-site-mismatched complexes with inefficient progression to the irreversible peptidyl transfer reaction.

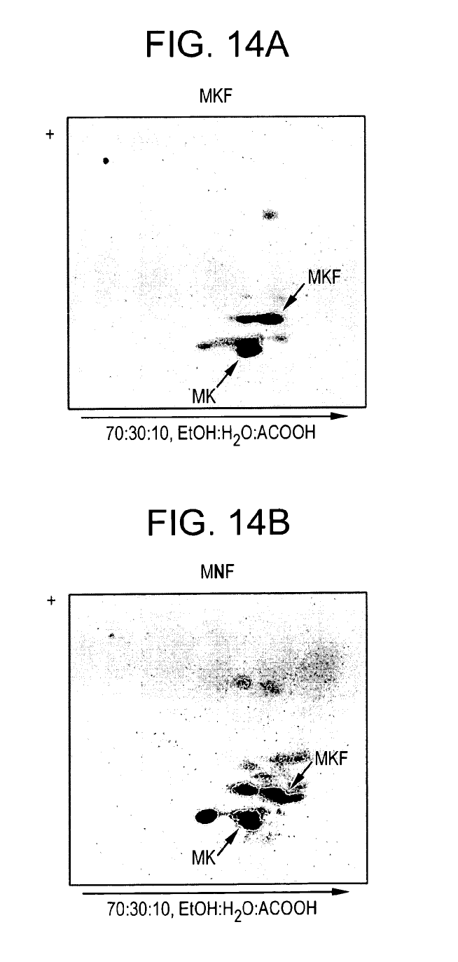

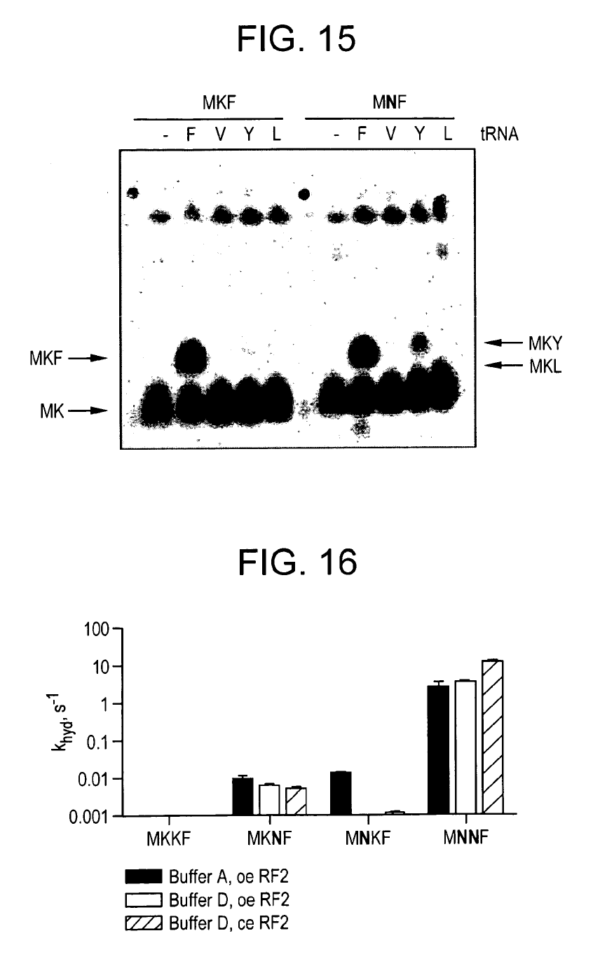

A more complete understanding of the observed competition from total tRNA mix comes from analysis of the actual peptide products. A next set of experiments compared product purity of two different sets of P-site-matched and mismatched complexes, MKI versus MNI, and MKF versus MNF, using a two-dimensional thin-layer chromatography (TLC) format. As anticipated on the basis of the high fidelity of protein synthesis, the P-site-matched complexes (MKI and MKF) yielded a predominant tripeptide product corresponding to the encoded sequence (FIG. 3b and FIG. 14). However, for both mismatched complexes (MNI and MNF) marked losses of fidelity were observed, such that a wide range of miscoded products is observed after the initial forced miscoding event. An estimate of partitioning to correct product (relative to partitioning to incorrect products) in the P-site-matched complex is higher than 90%, whereas for the mismatched complex that same value falls in a range between 10 and 30%. Indeed, it can be shown that specifically chosen near-cognate tRNAs react more readily with the mismatched complex than with the matched one (FIG. 15). The observed losses in fidelity for tRNA selection coupled with the promiscuity of release factors documented earlier, both after a simple miscoding event, suggest that the A site itself is generally perturbed.

### Example 4

### Amplification of Errors Leads to Fast Termination

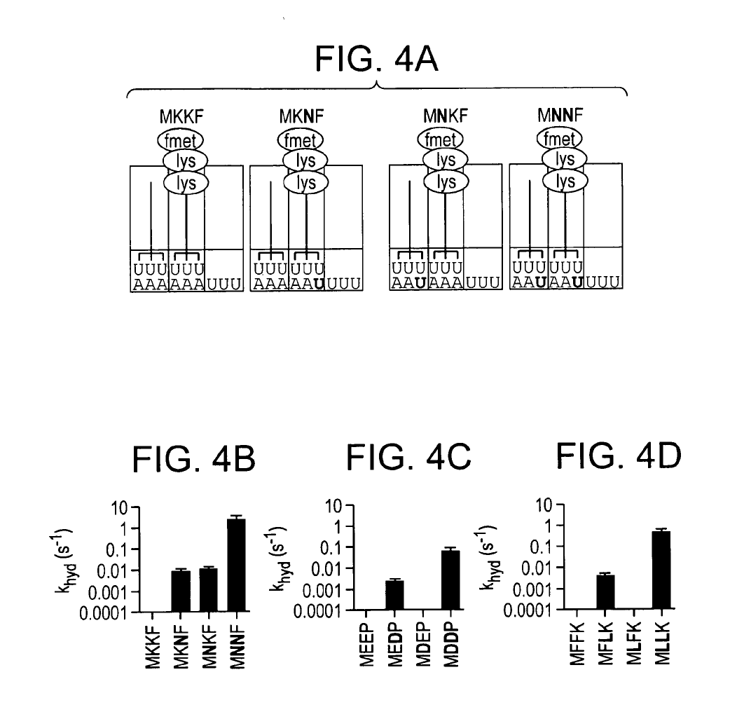

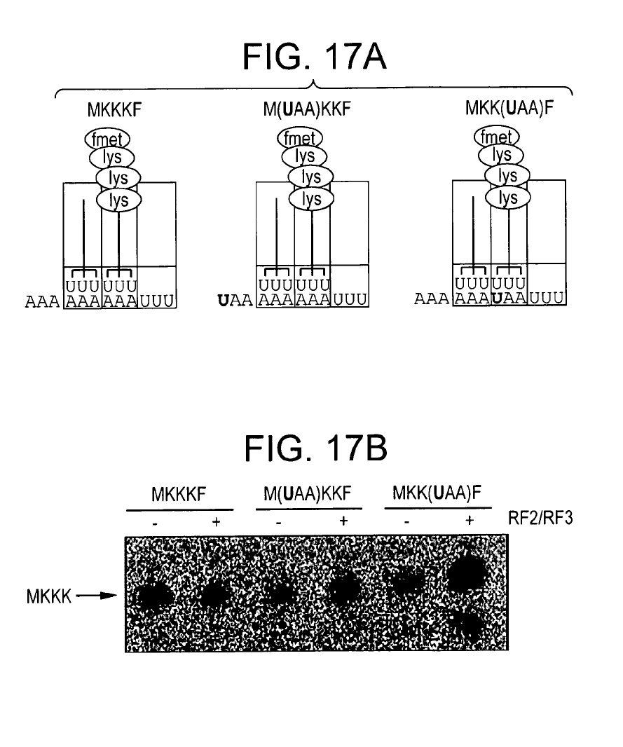

The previous sections have described a series of experiments suggesting that a single misincorporation during translation leads to marked changes in A-site behavior including increased mis coding and accelerated release of polypeptides on sense codons. Increasing the rate of miscoding is not terminal, but it does serve at a minimum to extend the window of opportunity for release factors to abort translation. It was next tested whether a second sequential miscoding event might generate a complex with even more unusual properties because it carries a mismatch in both the E- and P-site regions of the small ribosomal subunit. The consequences of the iterated miscoding were explored by programming tripeptidyl-tRNA ribosome complexes with a single mismatch in the P site (MKNF, MEDP or MFLK), single mismatch in the exit (E) site (MNKF, MDEP or MLFK), or a combination of both mismatches (MNNF, MDDP or MLLK) as might result from repeated miscoding events (the MKKF series is depicted in FIG. 4a). As seen previously, the P-site-mismatched complexes are robust substrates for RF2 and RF3, yielding rate con-stints for peptide release in the range of 0.005 to 0.01^{s-1}. For the singly mismatched E-site complexes, there was no stimulation of release activity in two of the complexes (MDEP and MLFK), whereas the results were buffer-dependent for the MNKF complex (FIG. 4b and FIG. 16). These differences are discussed herein. Most marked, however, was the increased rate of premature peptide release on each of the doubly mismatched complexes with rate constants for release ranging from 0.07 to 2 s⁻¹ (FIG. 4b-d). These values are in a range comparable to tRNA selection and thus should compete in the continuing process of protein synthesis. The robust nature of these findings was confirmed by observing very similar relative rates of release on the MKKF RNC series in a different butter (poly-mix, D) and with chromosomally-expressed (fully methylated) RF2 (FIG. 17). Control experiments were also performed to ensure that RNCs carrying a miscoding event that has fully exited the E site are no longer identified by RF2 as aberrant (FIG. 17).

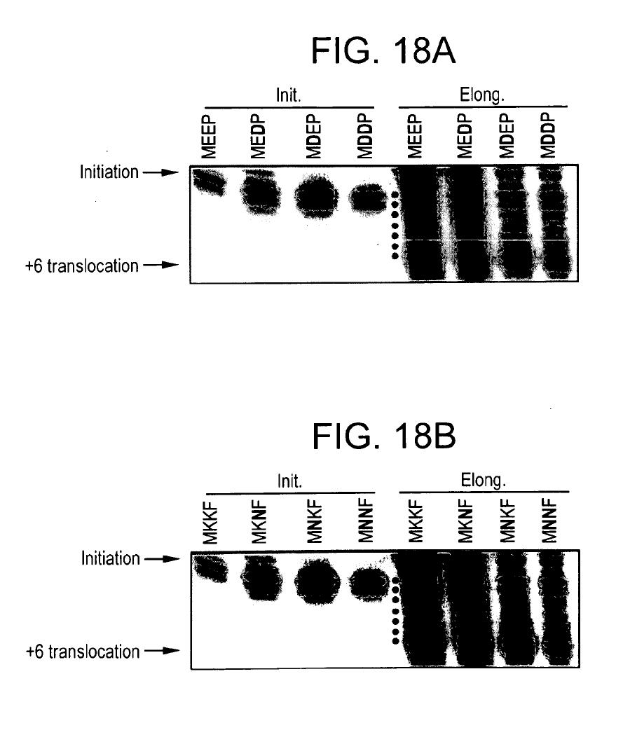

To further explore the molecular mechanism responsible for the disparate E-site effects, a toe-printing experiment was performed to determine the positioning of the tripeptidyl-tRNA RNCs on several mRNA species. For the MEEP series, the tripeptidyl-tRNA complexes seem rather uniform and fully extended with a toe-print positioned primarily at +6 relative to their starting position (FIG. 18a). Notably, for the MKKF series, whereas the P-site mismatched complex (MKNF) was positioned as anticipated on the mRNA, for both complexes carrying inn E-site mismatch (MNKF and MNNF), a substantial traction of the complex exhibits a heterogeneous banding pattern (FIG. 18b). These latter data are consistent with proposals suggesting that codon-anticodon interactions in the E site can be critical for reading frame maintenance during translation (23). It is suggested that the effects of E-site mismatches on the A site are mostly manifested through structural perturbations of the proximal P-site decoding helix, an idea nicely supported by studies in yeast demonstrating that P-site mismatches can affect frame maintenance (25).

### Example 5

### In Vivo Relevance of Post-Peptidyl Quality Control

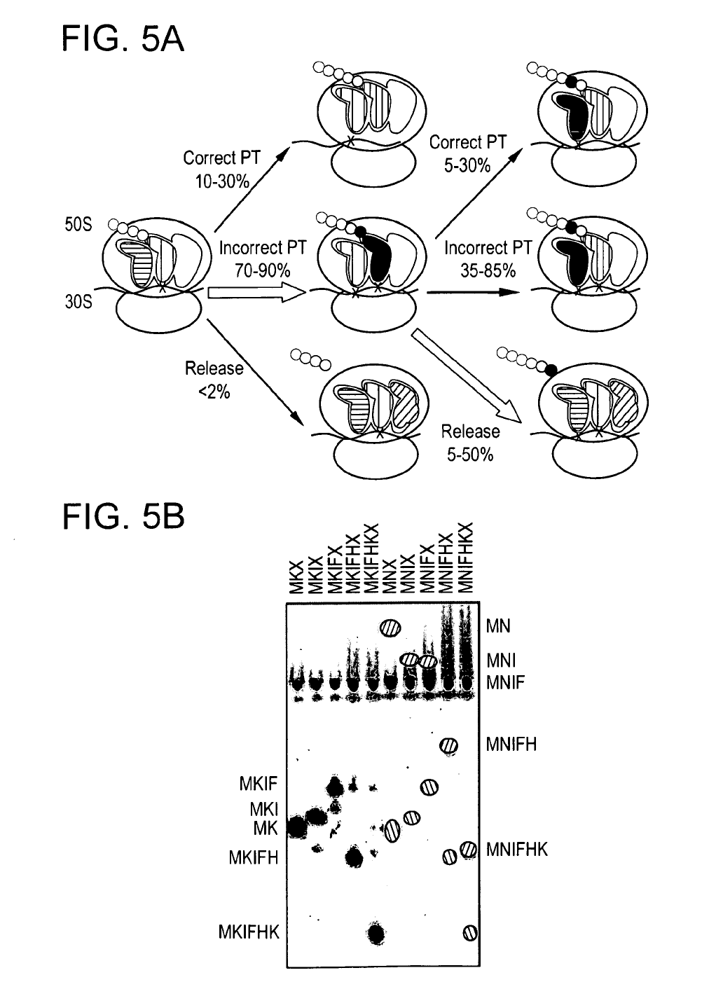

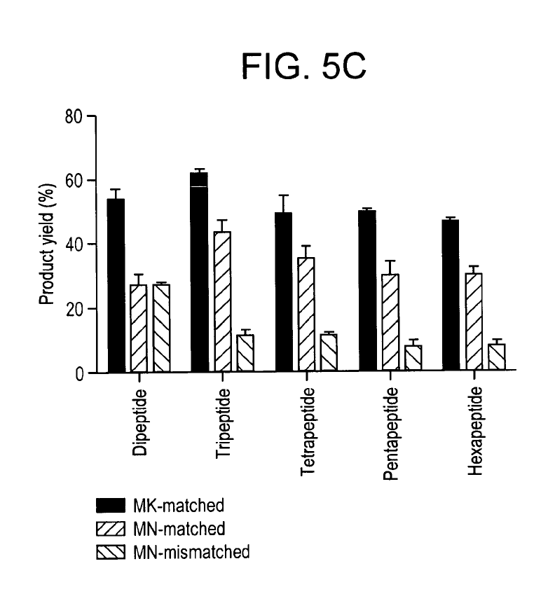

To evaluate the potential contribution of retrospective quality control to the fidelity of translation in the cell, the partitioning was estimated between premature release, inaccurate and accurate peptidyl transfer after a first miscoding event (FIG. 5a) on the basis of rough cellular estimates of tRNA and release-factor concentrations (50-200 uM and 6-25 μM, respectively, ref. 26) and the measured kₑₐₜ/Kₘ values of peptidyl transfer, incorrect peptidyl transfer and release after a single miscoding event (FIGS. 3, 4 and FIG. 8). It can be predicted from these calculations that the net effect of the iterated partitioning steps (detailed in FIG. 5a) is that a single initial miscoding event results in a marked increase in premature chain termination (highlighted by arrows).

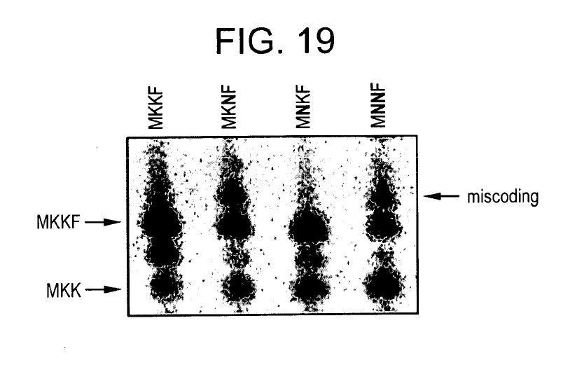

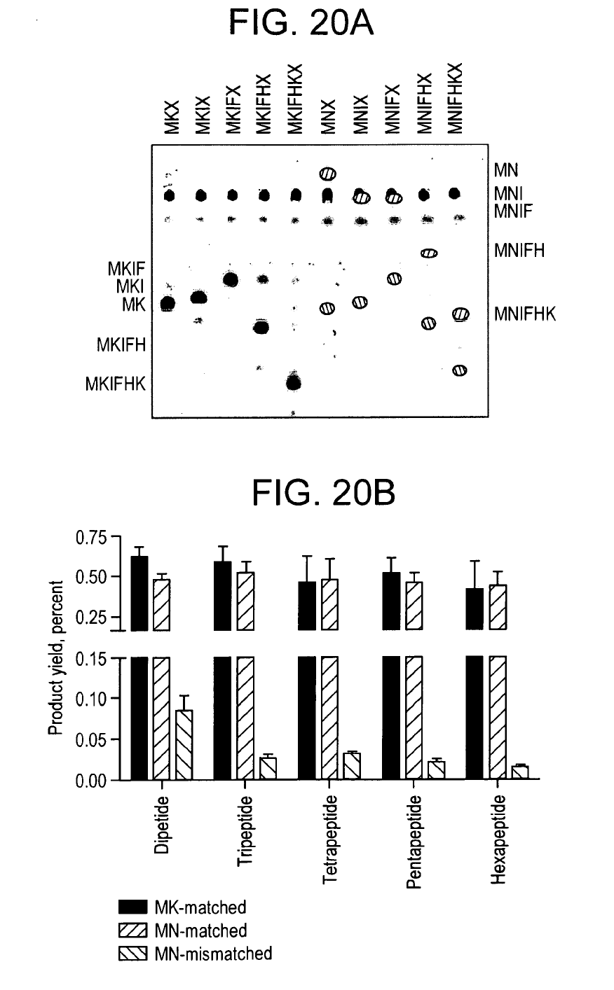

There are two predictions of the proposed model: (1) that the yield of full-length product will diminish after a miscoding event, and (2) that there will be evidence of prematurely truncated, multiply miscoded peptides. To test these predictions under competitive conditions, the translation of a heteropolymeric mRNA was evaluated sequence containing an AAU colon at position two for targeted decoding and miscoding by Asn-tRNA^{Asn} and Lys-tRNA^{Lys}, respectively, followed by sequence coding for abundant aminoacyl-tRNAs in the mixture. Translation of this mRNA in an S100 extract under limiting concentrations of Asn-tRNA^{Asn} allowed the consequences of cognate and near-cognate decoding to be followed during translation in an in-vivo-like setting. In this competition experiment (FIG. 5b, c), the yield of full-length product after an initial miscoding event was decreased nearly fourfold (relative to two different controls). When the experiment was repeated in buffer D, the observed reduction in product after a miscoding event was even inure impressive (tenfold, FIG. 20). This overall drop in yield is markedly consistent with the partitioning that was predicted in FIG. 5a. Although no strong signature of prematurely-released miscoded polypeptides is seen, a diffuse set of products would be very difficult to detect in the described TLC system. Experiments have also looked directly at partitioning for a defined (biochemically isolated) tripeptidyl tRNA RNC (MNNF) in the presence of S100 and aminoacyl-tRNA mix, and observe an extent of release consistent with estimates from the purified system (FIG. 19).

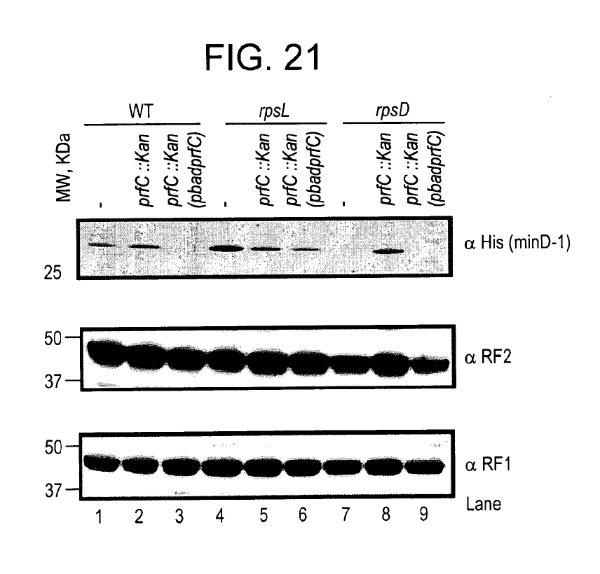

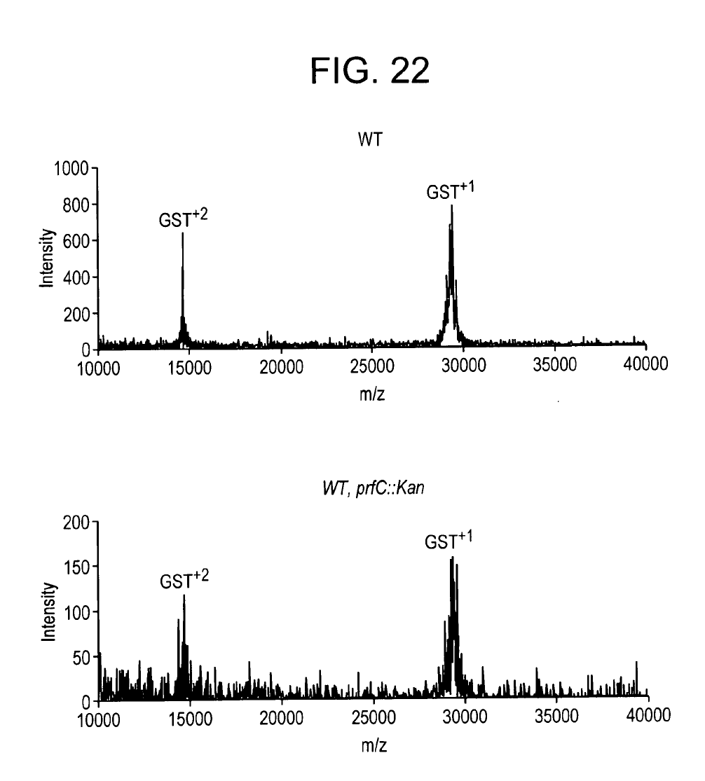

FIG. 21 shows Protein yield is increased when overexpressed in a restrictive E. coli strain. The archaeal protein (minD-1) was overexpressed in the indicated strains, where WT, rpsL (rpsL141) and rpsD (rpsD12) indicate wild-type, hyper-accurate (restrictive) and error-prone (ram) strains, respectively. prfC::Kan indicates a strain where the gene encoding RF3 was deleted by replacing it with a Kanamycin-resistance gene, whereas pbadprfC indicates that RF3 was overexpressed. FIG. 22 shows deterioration of protein quality in an RF3-deletion strain. Glutathione S transferase (GST) was isolated from the indicated strains, WT and RF3-deletion (prfC::Kan) strains.

A possible in vivo correlation for these studies comes from the known auto-regulation of RF2. Translation of RF2 in the cell depends on a programmed frameshifting event that occurs when a UGA codon encountered in the A site results in a stalled complex due to the low abundance of RF2 (29). As with many frameshifting events, the decoding helix found in the P site after the programmed frameshifting event is perturbed (with RF2, for example, a G•U mismatch in the first position of the codon) and thus should normally be a prime candidate for recognition by the surveillance system described here. Paradoxically, in this case, RF2 concentration is low and thus is riot likely to contribute to substantial premature chain termination until the overall RF2 levels in the cell are increased. It can be suggested that this feedback loop has evolved to effectively evade the quality control mechanism described here.

Quality control mechanisms are important throughout the cell in ensuring the faithful replication of the genuine and its expression into functional components. Like DNA replication, the quality control system that is described herein for protein synthesis depends on recognition of error after chemical incorporation of the building block into the growing polymer. However, unlike DNA replication, in which extension of the growing strand is completed after the hydrolytic action of the polymerase removes the misincorporated nucleotides, the quality control described here results in termination of protein synthesis.

In light of the competition experiments described earlier, it can be said that the post-peptidyl transfer process described here might contribute close to an order of magnitude to fidelity in vivo under standard conditions. Moreover, under conditions of starvation, in which amino acids become limiting in the cell and miscoding events are increasingly probable, it is suggested that this surveillance system could have an even more substantial rule in specifying the fidelity of translation. Experiments here were conducted on very early stage RNCs (almost initiation complexes) and that premature release might have a distinct rule during the elongation phase of translation. Nevertheless, the effects that were measured in this study are notable. To give some perspective, whereas false termination at a sense codon normally occurs with a frequency of less than once per 100,000 codons (measured here and in ref. 30) in fully matched complexes, in one of the doubly mismatched complexes, false termination occurs half the time. these changes in the biochemical activity of the ribosome are triggered by single mismatches positioned in the P and E sites of the small ribosomal subunit, highlighting the existence of another intricate molecular system within the ribosome that precisely dictates perfection in the transmission of the genetic code.

### Methods

The experiments described herein were performed with, but not limited to, the following methods and materials.

Materials. Buffers used were as follows: buffer A (50 mM Tris-HCl, pH 7.5, 70 mM NH4Cl, 30 mM KCl, 7 mM MgCl2, 1 mM dithiothreitol (DTT))31, buffer B (HiFi) (50 mM Tris-HCl, pH 7.5, 70 mM NH4Cl, 30 mM KCl, 3.5 mM MgCl2, 0.5 mM spermidine, 8 mM putrescine, and 2 mM DTT)₃, buffer C (polyamine) (20 mM HEPES-potassium-hydroxide, pH 7.6, 150 mM NH4Cl, 4.5 mM MgCl2, 2 mM spermidine, 0.05 mM spermine, 4 mM-mercaptoethanol)32, buffer D (polymix) (95 mM KCl, 5 mM NH4Cl, 5 mM magnesium acetate, 0.5 mM CaCl2, 8 mM putrescine, 1 mM spermidine, 5 mM potassium phosphate, pH 7.5, 1 mM DTT)10.

Escherichia coli MRE600 (ATCC29417) tight couple 70S ribosomes were prepared as described previously (33). Overexpressed native IF1 and IF3 and His-tagged IF2 were purified as described (34) Amino-terminally His-tagged RF1 and the 20 aminoacyl-tRNA synthetases were expressed and purified as previously described (35). His6-tagged EF-Tu and EF-G were purified over Ni-NTA resin, the His tag was later removed by tobacco etch virus protease, which was followed by a second passage over Ni-NTA column (36). Overexpressed His-tagged RF2 and RF3 were purified as described (15). Chromosomally-expressed RF2 was purified using a procedure similar to that previously described (15) except for the following modifications. After the ammonium sulphate precipitation following the first gel-filtration step, fractions containing RF2 were resuspended in 25 mM sodium phosphate, buffer pH 6.8, and dialysed against the same buffer overnight. The protein was then applied to a hydroxyapatite column (0.7 cm 5.2 cm), and eluted with a 50 ml linear phosphate gradient (25-500 mM). The purified protein was finally dialysed in a buffer comprised of 40 mM Tris-HCl, pH 7.5, 10 mM MgCl2, 100 mM KCl, 1 mM DTT and 50% glycerol.

tRNALys, tRNAPhe, tRNAfMet (all from E. coli) and rabbit muscle pyruvate kinase were purchased from Sigma-Aldrich. Total E. coli tRNA was purchased from Roche. mRNA templates were prepared from double-stranded DNA templates using run-off transcription by T7 RNA polymerase (37), and purified by PAGE. mRNAs used for dipeptidyl complexes had the following sequence: GGGUGUCUUGCGAGGAUAAGUGCAUU AUG (X) (Y) UGA UUUGCCCUUCUGUAGCCA, in which the initiator Met codon is in bold, whereas X and Y denote codons occupying the P and A site, respectively. The tripeptidyl RNCs were programmed with similar mRNAs that had an extra codon, Z, after the Y codon. The mRNA coding for fMet-Phe (AUG UUC) used for fidelity measurements (Supplementary FIG. 1) was chemically synthesized (Dharmacon).

tRNA charging. Aminoacylation and formylation of the initiator tRNAfMet with radiolabelled [35S]-methionine using an 5100 extract was performed as described previously (38). Pure tRNAs were charged by incubating the tRNA at 10 M with the corresponding aminoacyl-tRNA synthetase (1 M) in the presence of the appropriate amino acid and ATP (100 M and 2 mM, respectively) in the following buffer: 20 mM Tris-HCl, pH 7.5, 20 mM MgCl2, 1 mM DTT. After incubation at 37° C. for 30 min, the aminoacylated tRNA was purified by phenol and chloroform extraction followed by ethanol precipitation and resuspended in 20 mM potassium acetate buffer, pH 5.1, with 1 mM DTT. Total tRNA was charged using a similar procedure except that the tRNA concentration was increased to 100 M and all 20 aminoacyl-tRNA synthetases were added (1 M each) and all 20 amino acids were added (100 M each). In cases in which a single tRNA in the complete tRNA mixture was aminoacylated, the same basic reaction was set up, but only the desirable synthetase and amino acid were supplied (for example, for the Ile-tRNAIIe in FIG. 3a).

RNC ribosome complex formation. Initiation complexes were first prepared by incubating 70S ribosomes (2 M) with IF1, IF2, IF3, f[35S]Met-tRNAfMet (3 M each), and mRNA (6 M) in buffer C (or buffer D for experiments in Supplementary FIGS. 3, 11 and 15) in the presence of GTP at 2 mM at 37° C. for 45 min. RNCs were then obtained by adding equivalent volumes of initiation complexes and a pre-incubated elongation mixture containing EF-Tu (15 M), charged tRNA (6 M for dipeptidyl and 10 M for tripeptidyl complexes, respectively), EF-G (6 M), and GTP (2 mM) in buffer C (or buffer D, as above) and incubating at 37° C. for 10 min. Buffer C was used to form RNC complexes that were ultimately assayed in buffer A because of its permissivity in allowing near cognate tRNAs to react. Buffer A was considerably less promiscuous for certain near cognate pairings, thus making RNC complex formation difficult. To purify RNCs away from unincorporated tRNAs and factors, the reaction mixture was layered over a 1,3001 sucrose cushion (1.1 M sucrose, 20 mM Tris-HCl, pH 7.5, 500 mM NH4Cl, 10 mM MgCl2, 0.5 mM EDTA) and spun at 258,000 g in a TLA100.3 rotor for 2 h. The resulting pellet was resuspended in buffer A (or buffer D, as above), aliquoted and stored at −80° C. Electrophoretic TLC analysis of the complexes (see below) was used to determine the efficiency of dipeptide or tripeptide formation on the matched and mismatched mRNA templates. The typical yield for RNCs was as follows: in dipeptide- and tripeptide-matched complexes >80% of fMet was converted to the appropriate peptide; in mismatched complexes involving Lys-tRNALys or Glu-tRNAGlu the yield was >60% for dipeptides and >40% for tripeptides; whereas in mismatched complexes involving Phe-tRNAPhe, the yields were 40% and 10% for dipeptides and tripeptides, respectively. In addition, the amount of f[35S]Met that pellets provides further information about the stability of the RNC complexes. It is noted that mismatched templates typically yield less radioactivity in pelleting, probably because of the increased off-rates of the peptidyl-tRNA (Supplementary FIG. 4). The particular RNC of interest can be easily followed, despite the range in yield that is observed, because the relevant peptide product is well resolved in the electrophoretic TLC system (for example, FIG. 5b)

Release assays. Peptidyl RNCs (both dipeptidyl and tripeptidyl) at 25 to 150 nM were incubated with RF2 at 30 M (determined to be saturating for mismatched complexes, see Supplementary FIG. 3) in buffer A at 37° C. Where indicated, RF3 was added to a final concentration of 30 M with 2 mM GTP. Time points were obtained by taking aliquots at different time intervals and stopping the reaction with one-quarter of the volume of 25% formic acid. Released peptides of various lengths and identity were separated from unreacted peptidyl-tRNA using cellulose TLC plates that were electrophoresed in pyridine-acetate at pH 2.8 (ref. 21). Reactions with relatively fast rate constants (for example, >0.05 s−1), such as the doubly mismatched RNCs in FIG. 4, were performed on a quench-flow instrument (RQF-3 quench-flow, KinTek Corporation). The fraction of peptide released at each time point was quantified using ImageQuant v5.2 (Molecular Dynamics) and plotted against time. The data were fit to the first-order rate equation; F=Fmax (1−e-kt), in which F is the fraction hydrolyzed, to obtain the rate constant (k) and the fraction of the population (Fmax) that could react. In most cases, Fmax was found to be between 60% and 90%. To determine K1/2 values, release time courses were conducted with RNCs (25 nM) at varying concentrations of RF2 (0.025-30 M). The kobs values were obtained from individual fits at a given concentration of RF2 and the K1/2 was derived from the hyperbolic fit of the kobs versus [RF2] curve (Michaelis-Menten). The background rate—in the absence of RF2—was determined for all complexes and subtracted from the rate observed in the presence of RF2. For certain P-site-matched complexes, the rate of peptide release was immeasurably low, and so in these cases an upper limit for the rate constant is provided (for example, MKI and MKF complexes in FIG. 10.

Peptidyl transferase assays. EF-Tu at 100 M was first incubated with 2 mM GTP in buffer A for 15 min to promote exchange of the bound GDP for GTP. The enzyme was then diluted to 20 M in buffer A containing charged tRNA (10 M) and 2 mM GTP, and incubated for another 15 min. The mixture was incubated with an equal volume of RNC (final concentration 100 nM). The reaction was stopped by the addition of potassium hydroxide to a concentration of 100 mM. Reaction products were resolved by electrophoretic TLC as above, and analyzed similarly. Reactions performed with total tRNAs were carried out with final concentrations of EF-Tu and tRNA of 100 M and 80 M, respectively. The rate constants for these experiments were determined by fitting curves following the fraction of substrate (for example, dipeptide) that disappeared as a function of time.

Toe-print assay. Initiated and elongated peptidyl-tRNA complexes were prepared as above, except that the mRNAs used had extra sequence at the 3′-end to allow for an oligonucleotide primer to anneal and be extended by reverse transcriptase. The toe-printing reactions were then carried out essentially as described (39). The RNCs were resuspended in buffer A that was supplemented with an additional 10 mM MgCl2. A trace amount of 5′-radio labelled reverse transcription primer (5′ phosphorylated using polynucleotide kinase and [−32P]-ATP), and dNTPs (600 M each) were added. Primer extension was initiated by the addition of AMV reverse transcriptase at a concentration of 1 U 1-1. The reaction was incubated at 37° C. for 10 min, followed by the addition of sodium hydroxide at 100 mM and incubation at 90° C. for 10 min to digest the RNA. The reaction was ethanol precipitated before analysis on long format 6% PAGE.

Two-dimensional TLC separation. For resolution of complex peptidyl transfer reactions incubated with total tRNA mixtures, the peptidyl transfer reactions were performed essentially as described above. At the end of the reaction, peptidyl-tRNA was hydrolyzed with 100 mM KOH before spotting the sample on a 20 20 cm cellulose TLC. In the first dimension the mobile phase was composed of ethanol:water:acetic acid at a ratio of 70:20:10. The TLC was then thoroughly dried and run electrophoretically in pyridine-acetate buffer (pH 2.8) for the second dimension.

S100 in vitro translation. For FIG. 5b, purified initiation complexes, instead of the post-translocation complexes used in previous reactions, were prepared as described above, and then incubated (50 nM final concentration) with an 5100 extract containing 120 M tRNA (pre-charged with a tRNA synthetase mix lacking asparaginyl-tRNA synthetase (AsnRS), 2 mM GTP, 6 mM PEP and 0.02 mg ml-1 pyruvate kinase in buffer A at 37° C. for 10 min. For Supplementary FIG. 14, post-translocation tripeptidyl-RNCs were prepared as above, and reacted in a similar fashion with S100 and aminoacyl-tRNA mixture. The samples were resolved using the electrophoretic TLC system described above. Observed Asn-tRNAAsn activity in FIG. 5b probably derives from contaminating reagent that carries through in the bulk tRNA mixture or through impurities in the individual amino acids, as seen for Leu-tRNALeu activity in Supplementary FIG. 1.

Calculations for the model outlining contributions of the retrospective quality control to the fidelity of translation. During elongation, when an error is committed, the now mismatched translating RNCs will partition between three basic outcomes: 1) premature termination at a sense codon with RF2/RF3 (estimated by FIG. 2 to occur <2% of the time), 2) selection of the cognate tRNA and peptidyl transfer (estimated in FIG. 3b to occur 10-30% of the time), and 3) selection of another mismatched tRNA and peptidyl transfer (estimated in FIG. 3b to occur 70-90% of the time). The consequence of this most probable event (3) is the generation of a ribosome complex with mismatches in both the E and P sites. As shown in FIG. 4 and FIG. 17, these complexes are estimated to partition with high probability (˜5-50%) to abortive termination, while ˜50-95% of the population will dominantly partition such that another error in tRNA selection occurs. For these estimates, the measured Kₘ values are used for RF2 and tRNA on singly mismatched complexes (FIG. 9 and data not shown) of 1-2 μM, and in vivo concentration estimates of RF2 and individual tRNAs as essentially equivalent (5-20 μM). These equivalent values of Kₘ and cellular concentrations simplified the partitioning between release and PT (determined by multiplying the second order rate constants by the concentration) into the relative k_{cat} of the two processes (0.07-2 s−1 for release on a doubly mismatched complex, and 1-2 s−1 for PT when full competitor tRNA is added).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Edelmann, P. & Gallant, J. Mistranslation in E. coli. Cell 10, 131-137 (1977)
2. Bouadloun, F., Donner, D. & Kurland, C. G. Codon-specific missense errors in vivo. EMBO J. 2, 1351-1356 (1983)
3. Gromadski, K. B. & Rodnina, M. V. Kinetic determinants of high-fidelity tRNA discrimination on the ribosome. Mol. Cell. 13, 191-200 (2004)
4. Guth, E. C. & Francklyn, C. S. Kinetic discrimination of tRNA identity by the conserved motif 2 loop of a class II aminoacyl-tRNA synthetase. Mol. Cell. 25, 531-542 (2007)
5. Schmidt, E. & Schimmel, P. Mutational isolation of a sieve for editing in a transfer RNA synthetase. Science 264, 265-267 (1994)
6. Hopfield, J. J. Kinetic proofreading: a new mechanism for reducing errors in biosynthetic processes requiring high specificity. Proc. Natl. Acad. Sci. USA 71, 4135-4139 (1974)
7. Ninio, J. Kinetic amplification of enzyme discrimination. Biochimie 57, 587-595 (1975)
8. Soll, D. The accuracy of aminoacylation—ensuring the fidelity of the genetic code. Experientia 46, 1089-1096 (1990)
9. Szaflarski, W. et al. New features of the ribosome and ribosomal inhibitors: non-enzymatic recycling, misreading and back-translocation. J. Mol. Biol. 380, 193-205 (2008)
10. Jelenc, P. C. & Kurland, C. G. Nucleoside triphosphate regeneration decreases the frequency of translation errors. Proc. Natl. Acad. Sci. USA 76, 3174-3178 (1979)
11. Brutlag, D. & Kornberg, A. Enzymatic synthesis of deoxyribonucleic acid. 36. A proofreading function for the 3′ leads to 5′ exonuclease activity in deoxyribonucleic acid polymerases. J. Biol. Chem. 247, 241-248 (1972)
12. Precup, J. & Parker, J. Missense misreading of asparagine codons as a function of codon identity and context. J. Biol. Chem. 262, 11351-11355 (1987)
13. Brunelle, J. L., Shaw, J. J., Youngman, E. M. & Green, R. Peptide release on the ribosome depends critically on the 2′ OH of the peptidyl-tRNA substrate. RNA 14, 1526-1531 (2008)
14. Youngman, E. M., He, S. L., Nikstad, L. J. & Green, R. Stop codon recognition by release factors induces structural rearrangement of the ribosomal decoding center that is productive for peptide release. Mol. Cell. 28, 533-543 (2007)
15. Dincbas-Renqvist, V. et al. A post-translational modification in the GGQ motif of RF2 from Escherichia coli stimulates termination of translation. EMBO J. 19, 6900-6907 (2000)
16. Freistroffer, D. V., Kwiatkowski, M., Buckingham, R. H. & Ehrenberg, M. The accuracy of codon recognition by polypeptide release factors. Proc. Natl. Acad. Sci. USA 97, 2046-2051 (2000)
17. Baranov, P. V., Gesteland, R. F. & Atkins, J. F. Recoding: translational bifurcations in gene expression. Gene 286, 187-201 (2002)|
18. Hartz, D., McPheeters, D. S., Traut, R. & Gold, L. Extension inhibition analysis of translation initiation complexes. Methods Enzymol. 164, 419-425 (1988)
19. Brown, C. M., McCaughan, K. K. & Tate, W. P. Two regions of the Escherichia coli 16S ribosomal RNA are important for decoding stop signals in polypeptide chain termination. Nucleic Acids Res. 21, 2109-2115 (1993)
20. Katunin, V. I., Muth, G. W., Strobel, S. A., Wintermeyer, W. & Rodnina, M. V. Important contribution to catalysis of peptide bond formation by a single ionizing group within the ribosome. Mol. Cell. 10, 339-346 (2002)
21. Youngman, E. M., Brunelle, J. L., Kochaniak, A. B. & Green, R. The active site of the ribosome is composed of two layers of conserved nucleotides with distinct roles in peptide bond formation and peptide release. Cell 117, 589-599 (2004)
22. Freistroffer, D. V., Pavlov, M. Y., MacDougall, J., Buckingham, R. H. & Ehrenberg, M. Release factor RF3 in E. coli accelerates the dissociation of release factors RF1 and RF2 from the ribosome in a GTP-dependent manner. EMBO J. 16, 4126-4133 (1997)
23. Marquez, V., Wilson, D. N., Tate, W. P., Triana-Alonso, F. & Nierhaus, K. H. Maintaining the ribosomal reading frame: the influence of the E site during translational regulation of release factor 2. Cell 118, 45-55 (2004)
24. Geigenmuller, U. & Nierhaus, K. H. Significance of the third tRNA binding site, the E site, on E. coli ribosomes for the accuracy of translation: an occupied E site prevents the binding of non-cognate aminoacyl-tRNA to the A site. EMBO J. 9, 4527-4533 (1990)|
25. Sundararajan, A., Michaud, W. A., Qian, Q., Stahl, G. & Farabaugh, P. J. Near-cognate peptidyl-tRNAs promote +1 programmed translational frameshifting in yeast. Mol. Cell 4, 1005-1015 (1999)|
26. Dong, H., Nilsson, L. & Kurland, C. G. Co-variation of tRNA abundance and codon usage in Escherichia coli at different growth rates. J. Mol. Biol. 260, 649-663 (1996)
27. Manley, J. L. Synthesis and degradation of termination and premature-termination fragments of -galactosidase in vitro and in vivo. J. Mol. Biol. 125, 407-432 (1978)
28. Dong, H. & Kurland, C. G. Ribosome mutants with altered accuracy translate with reduced processivity. J. Mol. Biol. 248, 551-561 (1995)
29. Craigen, W. J. & Caskey, C. T. Expression of peptide chain release factor 2 requires high-efficiency frameshift. Nature 322, 273-275 (1986)
30. Jorgensen, F., Adamski, F. M., Tate, W. P. & Kurland, C. G. Release factor-dependent false stops are infrequent in Escherichia coli. J. Mol. Biol. 230, 41-50 (1993)
31. Pape, T., Wintermeyer, W. & Rodnina, M. Induced fit in initial selection and proofreading of aminoacyl-tRNA on the ribosome. EMBO J. 18, 3800-3807 (1999)|
32. Bartetzko, A. & Nierhaus, K. H. Mg2+/NH4+/polyamine system for polyuridine-dependent polyphenylalanine synthesis with near in vivo characteristics. Methods Enzymol. 164, 650-658 (1988)
33. Shaw, J. J. & Green, R. Two distinct components of release factor function uncovered by nucleophile partitioning analysis. Mol. Cell. 28, 458-467 (2007)
34. Brunelle, J. L., Youngman, E. M., Sharma, D. & Green, R. The interaction between C75 of tRNA and the A loop of the ribosome stimulates peptidyl transferase activity. RNA 12, 33-39 (2006)
35. Shimizu, Y. et al. Cell-free translation reconstituted with purified components. Nature Biotechnol. 19, 751-755 (2001)
36. Blanchard, S. C., Kim, H. D., Gonzalez, R. L., Puglisi, J. D. & Chu, S. tRNA dynamics on the ribosome during translation. Proc. Natl. Acad. Sci. USA 101, 12893-12898 (2004)
37. Zaher, H. S. & Unrau, P. J. T7 RNA polymerase mediates fast promoter-independent extension of unstable nucleic acid complexes. Biochemistry 43, 7873-7880 (2004)
38. Moazed, D. & Noller, H. F. Sites of interaction of the CCA end of peptidyl-tRNA with 23S rRNA. Proc. Natl. Acad. Sci. USA 88, 3725-3728 (1991)
39. Dorner, S., Brunelle, J. L., Sharma, D. & Green, R. The hybrid state of tRNA binding is an authentic translation elongation intermediate. Nature Struct. Mol. Biol. 13, 234-241 (2006)

1. A recombinant cell for overexpressing one or more proteins comprising one or more release factors.

2. The recombinant cell of claim 1, wherein the cell is prokaryotic.

3. The recombinant cell of claim 2, wherein the prokaryotic cell is an E. coli cell.

4. The recombinant cell of claim 1, wherein the cell is a eukaryotic cell.

5. The recombinant cell of claim 4, wherein the eukaryotic cell is selected from the group consisting of: Saccharomyces cerevisiae, Pichia pastoris and Baculovirus.

6. A recombinant restrictive cell for overexpressing one or more proteins comprising one or more release factors.

7. The recombinant restrictive cell of claim 6, wherein the cell is selected from the group consisting of: US157, UK285 and UK317.

8. The recombinant cell of claim, wherein the release factor is selected from the group consisting of: release factor 1 (RF1), release factor 2 (RF2), and release factor 3 (RF3).

9. The recombinant cell of claim 8, wherein the release factor is RF1.

10. The recombinant cell of claim 8, wherein the release factor is RF2.

11. The recombinant cell of claim 8, wherein the release factor is RF3.

12. The recombinant cell of claim 9, wherein RF1 corresponds to SEQ ID NO: 1.

13. The recombinant cell of claim 10, wherein RF2 corresponds to SEQ ID NO: 2.

14. The recombinant cell of claim 11, wherein RF3 corresponds to SEQ ID NO: 3.

15. The recombinant cell of claim 8, wherein the release factor is RF2 and RF3.

16. A recombinant cell for overexpressing one or more proteins comprising RF3, or
A recombinant restrictive cell for overexpressing one or more proteins comprising RF3, or
A recombinant cell for overexpressing one or more proteins comprising RF1, or
A recombinant restrictive cell for overexpressing one or more proteins comprising RF1, or
A recombinant cell for overexpressing one or more proteins comprising RF2, or
A recombinant restrictive cell for overexpressing one or more proteins comprising RF2, or
A recombinant cell for overexpressing one or more proteins comprising RF2 and RF3, or
A recombinant restrictive cell for overexpressing one or more proteins comprising RF2 and RF3.

17-23. (canceled)

24. The recombinant cell of claim 1, further comprising a vector comprising a codon optimized gene encoding the protein.

25-27. (canceled)

28. A method for producing a recombinant protein comprising:
culturing the cell of claim 1 under conditions such that one or more proteins are overexpressed;
isolating the expressed recombinant protein.

29. A method for producing a recombinant protein comprising:
culturing the cell of claim 5 under conditions such that one or more proteins are overexpressed;
isolating the expressed recombinant protein.

30-33. (canceled)

34. A method for producing a recombinant protein comprising:
transfecting or transforming a cell comprising one or more release factors with a gene encoding the recombinant protein;
culturing the cell;
isolating the expressed recombinant protein.

35-44. (canceled)

45. A recombinant protein produced by the method of claim 28.
